(19) United States
(12) Patent Application Publication
(10) Pub. No.: US 2018/0303753 A1
(43) Pub. Date: Oct. 25, 2018

(54) TARGETED DELIVERY OF SPRAY-DRIED FORMULATIONS TO THE LUNGS

(71) Applicants: Keith Try UNG, San Carlos, CA (US); Jeffry G. WEERS, San Carlos, CA (US); Daniel HUANG, San Carlos, CA (US); Nagaraja RAO, San Carlos, CA (US); Yoen-Ju SON, San Carlos, CA (US)

(72) Inventors: Keith Try UNG, San Carlos, CA (US); Jeffry G. WEERS, San Carlos, CA (US); Daniel HUANG, San Carlos, CA (US); Nagaraja RAO, San Carlos, CA (US); Yoen-Ju SON, San Carlos, CA (US)

(21) Appl. No.: 15/758,643

(22) PCT Filed: Sep. 7, 2016
(86) PCT No.: PCT/IB2016/055331

§ 371 Date: Mar. 8, 2018

Related U.S. Application Data
(60) Provisional application No. 62/215,904, filed on Sep. 9, 2015.

(51) Int. Cl. A61K 9/00; A61K 9/16

(52) U.S. Cl. CPC A61K 9/0075; A61K 38/2221; A61K 9/1682; A61K 9/1617

(57) ABSTRACT

Disclosed are inhalation formulations of dry powders comprising particles and processes which yield particles to effectively bypass unwanted deposition in the mouth and throat. Embodiments of the invention are characterized by an inertial parameter which provide an in vitro total lung dose of greater than 80% of a nominal dose. Embodiments of formulations include neat formulations containing active agent only; formulations of active agent and buffer; and formulations comprising active agent, a glass-forming, and/or a shell-forming excipient. Also provided are methods for making the dry powder formulations. The powder formulations are useful for the treatment of diseases and conditions especially respiratory diseases and conditions.

### FIELD OF THE INVENTION

The invention relates to inhalation formulations of dry powders comprising particles and processes for delivering the powder formulations which enable the particles to effectively bypass unwanted deposition in the mouth and throat, thus increasing total lung dose (TLD) in vitro. Embodiments of the invention are characterized by an inertial parameter which provides an in vitro total lung dose (TLD) of greater than 80% of a nominal dose. Embodiments of formulations include neat formulations containing active agent only; formulations of active agent and buffer; and formulations comprising active agent, a buffer, a glass-forming, and/or a shell-forming excipient. Also provided are methods for making the dry powder formulations of the present invention. The powder formulations are useful for the treatment of diseases and conditions especially respiratory diseases and conditions.

### BACKGROUND

Targeted drug delivery may be defined as a method for delivering medication to a patient in a manner that increases the concentration of the medication in some parts of the body relative to others. For medications administered via oral inhalation, improved lung targeting may be desired, and may achieved, in part, by minimizing deposition in the oropharynx (i.e., the mouth and throat; collectively also referred to as the upper respiratory tract, or URT). Unwanted deposition in the oropharynx can lead to higher drug doses, increases in systemic levels of drug (for drugs that are orally bioavailable), and in some instances increases in local and systemic side effects (e.g., as with inhaled corticosteroids).

For drugs with poor oral bioavailability and a desired site of action in the systemic circulation (e.g., many peptides and proteins), improved targeting of drug to the lungs, and in particular to the alveoli enables improvements in systemic bioavailability. An ability to more effectively target drug to the lungs may also enable larger doses to be delivered from a given sized powder receptacle (i.e., less drug wastage).

Deposition of inhaled powders in the oropharynx is governed by inertial impaction, with deposition proportional to the inertial parameter (dₐ²Q), where dₐ is the aerodynamic diameter and Q is the volumetric flow-rate achieved by subjects through a dry powder inhaler.

The aerodynamic diameter depends both on the geometric diameter (d_{g}) and density (ρₚ) of the particles, that is:

dₐ=d_{g}\sqrt{ρₚ} (Equation 1)

For a single particle, deposition in the oropharynx will be reduced with decreases in dₐ, d_{g} and ρₚ. For an ensemble of particles the story is more complex, as bulk powders exist, in part, as agglomerates of particles that must be dispersed to primary particles or to agglomerates of particles that are fine enough to enable efficient delivery to the lungs (i.e., “respirable agglomerates”). Delivery of dry powder aerosols to the lungs depends on interplay between formulation and device. The ability to effectively fluidize and disperse dry powder agglomerates is dependent on the ratio of interparticle cohesive forces present in the powder, to the hydrodynamic forces (e.g., drag and lift forces) generated in the dry powder inhaler. At relative humidities less than 60%, interparticle cohesive forces are dominated by van der Waals interactions.

For rigid spheres, van der Waals forces (F_{vdw}) are directly proportional to d_{g} and the Hamaker constant (A), and inversely proportional to the square of the separation distance (r), that is:

$${F}_{vdw} = \frac{{Ad}_{g}}{24 {r}^{2}} ( rigid spheres ) \qquad ( Equation 2 )$$

In contrast, drag and lift forces scale with d_{g}². As d_{g} decreases into sizes required for efficient delivery into the lungs (e.g., d_{g}=1-5 μm), cohesive forces typically are larger than the hydrodynamic forces resulting in powders that are poorly dispersed.

Particle engineering strategies may be utilized to minimize interparticle cohesive forces via control of the surface composition and morphology of particles. In this regard, spray drying is a bottom-up manufacturing process that enables production of micron-sized particles, with control of the surface composition and micromeritic properties of the particles, for example size, density, porosity, and surface roughness (i.e., rugosity).

Spray dried proteins, such as insulin, may adopt a corrugated (i.e., raisin-like) particle morphology with a high rugosity provided they are dried rapidly. The protuberances forming the corrugations, called asperities, typically have a small radius of curvature (<0.1 μm). The mean van der Waals force depends strongly on the surface structure of the particles, i.e., the size distribution of the asperities and their surface density. To calculate the van der Waals force for corrugated particles with high surface asperity densities, it has been proposed to not use d_{g} in Equation 2, but instead to use an effective diameter (d_{eff}), given by the diameter of the asperities. Under these conditions, the van der Waal's forces can be several orders of magnitude lower than is observed for micron-sized solid spheres.

Improvements in respirable fraction (that is, particles having a dₐ<5 μm) have been demonstrated for spray-dried particles as the morphology is altered to increase surface roughness or corrugation. Nonetheless, significant deposition in the oropharynx (≥30%) is still observed.

Current marketed dry powder inhalation products comprising lactose blends or spheronized particles typically achieve a total lung dose in vivo between about 10% and 30% of the nominal dose. Exubera® and TOBI® Podhaler™, the first marketed dry powder products based on spray-drying, achieve a total lung dose in vivo of approximately 40% and 60%, respectively.

Therefore, it is desirable to provide spray-dried particles for inhalation which provide one or more advantages of: being more effectively targeted to the lungs; providing a high total lung dose; and effectively bypassing deposition in the oropharynx.

### SUMMARY

Embodiments of the invention comprise a carrier-free pharmaceutical composition deliverable from a dry powder inhaler, comprising active agent, wherein an in vitro total lung dose is greater than 90% of a delivered dose, or greater than 80% of a nominal dose, or both, and wherein the particles in the delivered dose have an inertial parameter between 120 and 400 μm² L/min.

Embodiments of the invention comprise a carrier-free pharmaceutical composition deliverable from a dry powder inhaler, the composition comprising a plurality of particles, comprising: a core comprising an active agent and at least one glass forming excipient, and a shell comprising hydrophobic excipient and a buffer; and wherein an in vitro total lung dose is greater than 90% w/w of the delivered dose, or greater than 80% of a nominal dose, or both.

A carrier-free pharmaceutical composition comprising a plurality of primary particles and particle agglomerates deliverable from a dry powder inhaler, the composition comprising active agent, and wherein an in vitro total lung dose (TLD) is greater than 90% of a delivered dose, or greater than 80% of a nominal dose, or both, and wherein the primary particles are characterized by a corrugated morphology, a median aerodynamic diameter (Dₐ) between 0.3 and 1.0 μm, and wherein the particles and particle agglomerates delivered from a dry powder inhaler have a mass median aerodynamic diameter (MMAD) between 1.0 and 3.0 μm.

Embodiments of the invention comprise a powder pharmaceutical composition deliverable from a dry powder inhaler, comprising particles comprising active agent, wherein an in vitro total lung dose is greater than 90% w/w of the delivered dose, and wherein the composition comprises at least one characteristic of being carrier-free, a particle density of 0.05 to 0.3 g/cm³; a particle rugosity of 3 to 20; particles made by a process comprising spray drying from an ethanol:water mixture; and particles made by a process comprising spray drying from an ethanol:water mixture having an ethanol:solids ratio of between 1 and 20.

Embodiments of the invention comprise a method of delivering to the lungs of a subject particles comprising a dry powder, the method comprising: preparing a solution of an active agent in a water/ethanol mixture, wherein the ethanol is present between 5 and 20%, spray drying the solution to obtain particulates, wherein the primary particulates are characterized by a particle density of between about 0.05 and 0.3 g/cm³ a geometric diameter of 1.0-2.5 microns and an aerodynamic diameter of 0.3-1.0 microns; packaging the spray-dried powder in a receptacle; providing an inhaler having a means for extracting the powder for the receptacle, the inhaler further having a powder fluidization and aerosolization means, the inhaler operable over a patient-driven inspiratory effort of about 2 to about 6 kPa; the inhaler and powder together providing an inertial parameter of between about between 120 and 400 μm² L/min and wherein the powder, when administered by inhalation, provides at least 90% lung deposition.

Embodiments of the invention comprise a method of preparing a dry powder medicament formulation for pulmonary delivery, comprising preparing a solution of an active agent in a water/ethanol mixture, wherein the ethanol is present between 5 and 20%, and spray drying the solution to obtain particulates, wherein the primary particulates are characterized by a particle density of between about 0.05 and 0.3, a geometric diameter of 1.0-2.5 microns and an aerodynamic diameter of 0.3-1.0 microns.

Embodiments of the invention comprise a dry powder formulation comprising particulates which provide an in vitro total lung dose (TLD) of between 80% and 100% weight/weight (w/w) of the nominal dose, for example between 85% and 95% w/w.

Embodiments of the invention comprise a dry powder formulation comprising particulates which provide an in vitro TLD of between 90% and 100% w/w of the delivered dose, for example between 90% and 99% w/w.

Embodiments of the invention comprise a dry powder formulation comprising particulates which provide an in vitro total lung dose (TLD) of between 80% and 100% weight/weight (w/w) of the nominal dose, or between 90% and 100% w/w of the delivered dose, or both.

Embodiments of the invention provide a dry powder formulation comprising particulates comprising a delivered dose wherein the particulates are characterized by an inertial impaction parameter (dₐ²Q) of between 120 and 400 μm² L/min, for example between 150 and 300 μm² L/min.

Embodiments of the invention comprise a dry powder formulation comprising particulates characterized by one or more micromeritic properties (e.g., d_{g}, dₐ, ρₚ, rugosity) and by one or more process parameters (e.g., particle population density, ethanol/solids ratio) which achieve a TLD between 80% and 95% w/w of the nominal dose, and/or between 90% and 100% w/w of the delivered dose.

Embodiments of the invention incorporate TLD, dₐ²Q, Dₐ, and MMAD to define a new region of particle space, which provide a significant improvement in lung targeting and dose consistency. Dₐ may be calculated from the ×50 and from the tapped density. Embodiments of the invention comprise process parameters directed to lowering ×50 and tapped density to enable small Dₐ values (on the order of less than 700 nm).

### Terms

Terms used in the specification have the following meanings:

“Active ingredient”, “therapeutically active ingredient”, “active agent”, “drug” or “drug substance” as used herein means the active ingredient of a pharmaceutical, also known as an active pharmaceutical ingredient (API).

“Fixed dose combination” as used herein refers to a pharmaceutical product that contains two or more active ingredients that are formulated together in a single dosage form available in certain fixed doses.

“Carrier-free” formulations as used herein refer to formulations which do not contain carrier particles in an interactive mixture with micronized drug particles. In typical lactose blends, the carrier particles are comprised of coarse lactose monohydrate carrier particles with a geometric diameter between 60 and 200 μm. As such, any drug particles which remain adhered to the carrier particles will not be respirable, and will deposit in the device and/or upper respiratory tract during inhalation.

“Extrafine” formulations are defined as having aerodynamic particle size distributions that target the small airways. Such formulations typically have a mass median aerodynamic diameter less than about 2 μm.

“Amorphous” as used herein refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically a second order phase transition (“glass transition”).

“Crystalline” as used herein refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically a first order phase transition (“melting point”). In the context of the present invention, a crystalline active ingredient means an active ingredient with crystallinity of greater than 85%. In certain embodiments the crystallinity is suitably greater than 90%. In other embodiments the crystallinity is suitably greater than 95%.

“Drug Loading” as used herein refers to the percentage of active ingredient(s) on a mass basis in the total mass of the formulation.

“Mass median diameter” or “MMD” or “×50” as used herein means the median diameter of a plurality of particles, typically in a polydisperse particle population, i.e., consisting of a range of particle sizes. MMD values as reported herein are determined by laser diffraction (Sympatec Helos, Clausthal-Zellerfeld, Germany), unless the context indicates otherwise. In contrast, d_{g} represents the geometric diameter for a single particle.

“Tapped densities” or ρ_{tapped}, as used herein were measured according to Method I, as described in USP <616>Bulk Density and Tapped Density of Powders. Tapped densities represent the closest approximation of particle density, with measured values that are approximately 20% less than the actual particle density.

“Puck densities” as used herein represent the bulk density of powder measured at a specified level of compression. For the purposes of this invention, the puck densities were determined at a vacuum suction pressure of 81 kPa.

“Rugous” as used herein means having numerous wrinkles or creases, i.e., being ridged or wrinkled.

“Rugosity” as used herein is a measure of the surface roughness of an engineered particle. For the purposes of this invention, rugosity is calculated from the specific surface area obtained from BET measurements, true density obtained from helium pycnometry, and the surface to volume ratio obtained by laser diffraction (Sympatec), viz:

Rugosity=(SSA·ρₜᵣᵤₑ)/Sᵥ

where Sᵥ=6/D₃₂, where D₃₂ is the average diameter based on unit surface area. Increases in surface roughness are expected to reduce interparticle cohesive forces, and improve targeting of aerosol to the lungs. Improved lung targeting is expected to reduce interpatient variability, and levels of drug in the oropharynx and systemic circulation. In one or more embodiments, the rugosity Sᵥ is from 3 to 20, e.g., from 5 to 10.

“Median aerodynamic diameter of the primary particles” or Dₐ as used herein, is calculated from the mass median diameter of the bulk powder as determined via laser diffraction (×50) at a dispersing pressure sufficient to create primary particles (e.g., 4 bar), and their tapped density, namely: Dₐ=×50 (ρ_{tapped})^{1/2}.

“Mass median aerodynamic diameter” or “MMAD” as used herein refer to the median aerodynamic size of a plurality of particles, typically in a polydisperse population. The “aerodynamic diameter” is the diameter of a unit density sphere having the same settling velocity, generally in air, as a powder and is therefore a useful way to characterize an aerosolized powder or other dispersed particle or particle formulation in terms of its settling behaviour. The aerodynamic particle size distributions (APSD) and MMAD are determined herein by cascade impaction, using a NEXT GENERATION IMPACTOR™. In general, if the particles are aerodynamically too large, fewer particles will reach the deep lung. If the particles are too small, a larger percentage of the particles may be exhaled. In contrast, dₐ represents the aerodynamic diameter for a single particle.

“Nominal Dose” or “ND” as used herein refers to the mass of drug loaded into a receptacle (e.g., capsule or blister) in a non-reservoir based dry powder inhaler. ND is also sometimes referred to as the metered dose.

“Delivered Dose” or “DD” as used herein refers to an indication of the delivery of dry powder from an inhaler device after an actuation or dispersion event from a powder unit. DD is defined as the ratio of the dose delivered by an inhaler device to the nominal or metered dose. The DD is an experimentally determined parameter, and may be determined using an in vitro device set up which mimics patient dosing. DD is also sometimes referred to as the emitted dose (ED).

“Total Lung Dose” (TLD) as used herein, refers to the percentage of active ingredient(s) which is not deposited in the Alberta Idealized Throat (AIT), and instead is captured on a filter post-throat, following inhalation of powder from a dry powder inhaler at a pressure drop of 4 kPa. The AIT represents an idealized version of the upper respiratory tract for an average adult subject. The 4 kPa pressure drop was selected in order to standardize how the measurement of TLD is performed, in much the same way that a 4 kPa pressure drop is generally used in measurement of MMAD or DD. A 4 kPa pressure drop represents the median pressure drop achieved by subjects following comfortable inhalation with a dry powder inhaler. Data can be expressed as a percentage of the nominal dose or the delivered dose. Unless otherwise stated, TLD is measured in the AIT model; and unless otherwise stated, measured at a 4 kPa pressure drop. Information on the AIT and a detailed description of the experimental setup can be found at: www.copleyscientific.com.

“Inertial parameter” as used herein refers to the parameter which characterizes inertial impaction in the upper respiratory tract. The parameter was derived from Stoke's Law and is equal to dₐ²Q, where dₐ is the aerodynamic diameter, and Q is the volumetric flow rate.

“Solids Content” as used herein refers to the concentration of active ingredient(s) and excipients dissolved or dispersed in the liquid solution or dispersion to be spray-dried.

“ALR” as used herein is a process parameter defining the air to liquid ratio utilized in an atomizer. Smaller ALR values typically produce larger atomized droplets.

“Particle Population Density” (PPD) as used herein is a dimensionless number calculated from the product of the solids content and the atomizer liquid flow rate divided by the total dryer gas flow rate. The PPD has been observed to correlate with primary geometric particle size.

“Primary particles” refer to the smallest divisible particles that are present in an agglomerated bulk powder. The primary particle size distribution is determined via dispersion of the bulk powder at high pressure and measurement of the primary particle size distribution via laser diffraction. A plot of size as a function of increasing dispersion pressure is made until a constant size is achieved. The particle size distribution measured at this pressure represents that of the primary particles.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

Unless otherwise stated, or clear from the context, numerical ranges include both the endpoints and any value therebetween.

The entire disclosure of each United States patent and international patent application mentioned in this patent specification is fully incorporated by reference herein for all purposes.

### DESCRIPTION OF THE DRAWINGS

The dry powder formulation of the present invention may be described with reference to the accompanying drawings. In those drawings:

FIG. 1 is a series of curves that represent various deposition fractions in the upper respiratory tract. Each deposition fraction correlates with an inertial parameter, dₐ²Q. The curves represent the range of flow rates (Q) and aerodynamic diameters (dₐ) that result in the targeted value of dₐ²Q. The shaded area represents the range of flow rates achievable with portable dry powder inhalers, including the Concept1 (C1) and Simoon (S) devices.

FIGS. 2A-2F are scanning electron microscopic (SEM) images of spray-dried insulin powders under different formulation and/or processing conditions.

FIG. 3 is a graph showing the impact of the ethanol/total solids ratio on bulk density for spray-dried insulin powders.

FIG. 4 is a graph showing the impact of the particle population density (PPD) on primary particle size for spray-dried insulin powders.

FIG. 5 is a graph showing the TLD as a function of the calculated aerodynamic diameter of the primary particles for spray dried formulations comprising a monoclonal antibody fragment and a protein (RLX030).

### DETAILED DESCRIPTION

Embodiments of the present invention are directed to a formulation and process to improve the lung targeting of amorphous APIs in a solution-based spray drying process.

Maintaining acceptable powder fluidization and dispersion for spray-dried powders dictates that in some embodiments primary particles have a mass median geometric diameter in the micron-size range (×50=1.0 to 2.5 microns). However, enabling dose delivery for all particles to the lungs dictates that both primary particles and their particle agglomerates be respirable. This requires that the primary particles should have an aerodynamic diameter in the nanometer size range (Dₐ=200 to 700 nm). In order to achieve this end, the particles in preferred embodiments are carrier-free with a corrugated morphology and low tapped density (or ρ_{tapped}=0.03 to 0.3 g/cm³). Overall, all of the particles in the DD should have an MMAD in the range from about 1.5 to 3 microns.

### Formulation/Particle Engineering

Embodiments of the present invention provide a dry powder formulation comprising spray-dried particles and agglomerates of spray-dried particles that effectively bypass deposition in the oropharynx of an average adult subject, enabling targeted delivery of medicament into the lungs.

Embodiments of the present invention provide particles of a dry powder formulation of the invention which suitably have an in vitro total lung dose (TLD) of between 80 and 95% w/w of the nominal dose, for example between 85 and 90% w/w for an average adult subject.

Embodiments of the present invention provide particles of a dry powder formulation of the invention which suitably have an in vitro total lung dose (TLD) of between 90 and 100% w/w of the delivered dose, for example between 90 and 99% w/w, or any value therebetween, for an average adult subject.

In order to achieve a TLD of 100% or nearly 100%, all of the particles and particle agglomerates must bypass deposition in the oropharynx. This is not possible with traditional carrier-based formulations comprising an ordered mixture of coarse lactose carrier particles and micronized drug. In carrier-based formulations, drug that remains adhered to the carrier particles is not respirable, and instead is deposited in the oropharynx. Of course, the present invention is not limited to embodiments which result in 100% TLD; rather formulations which provide the noted high levels of TLD and/or described functional results are within the scope of the present invention.

Embodiments of the dry powder formulation of the present invention comprise carrier-free formulations, where the carrier-free particles are manufactured using a bottom-up solution-based spray-drying process. Important, in some embodiments of the invention, to achieving targeted delivery with a TLD>90% is the need for any agglomerates of particles to also have a suitably low inertial parameter.

Embodiments of the dry powder formulation of the present invention comprising the delivered dose suitably have an inertial parameter (dₐ²Q) of between 120 and 400 μm² L/min, for example between 125 and 375, or 130 and 350, or 140 and 325, or 150 and 300, all measured as μm² L/min.

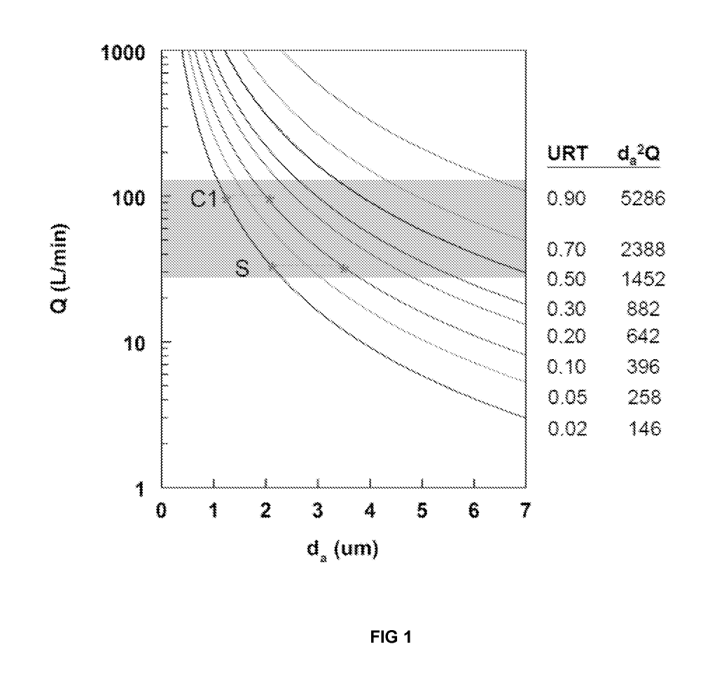

FIG. 1 is a plot of exemplary combinations of Q and dₐ needed to achieve a given dₐ²Q value, which correlates with a measured deposition fraction in the oropharynx (i.e., URT) according to the empirical equation derived by Stahlhofen et al. for monodisperse liquid aerosols (J Aerosol Med. 1989, 2:285-308). The bottom curve on the plot (dₐ²Q=146) leads to 2% deposition of particles in the oropharynx. This can, in principle, be achieved via various combinations of Q and dₐ. For example, the curve predicts that 2% deposition in the oropharynx (98% lung dose) occurs for dₐ=7 μm, provided that Q=3 L/min. Similarly, the curve predicts 2% oropharyngeal deposition (98% lung dose) for dₐ=0.5 μm, provided that Q=1000 L/min. Neither of the values of Q are presently practical for inhalation by subjects with portable dry powder inhalers. The grayed portion of the curve represents the range of Q values that is achievable with present dry powder inhalers. This places a practical limit on the upper end of acceptable dₐ values. In order to achieve 98% or greater lung dose, dₐ must be about 2.0 μm or less. In order to achieve 90% lung dose, dₐ can be as large as about 3.5 μm, depending on the nature of the device.

A dry powder inhaler is classified in terms of its resistance to airflow: low, medium and high resistance devices have resistances of ≤0.07, 0.08-0.12, and ≥0.13 cm H₂O^{0.5}/L/min, respectively. For a high resistance inhaler (e.g., Novartis' Simoon inhaler (R=0.19 cm H₂O^{0.5}/L/min)—designated as S on the curve), the value of Q at a patient effort comprising a 4 kPa pressure drop is significantly lower than for a low resistance device (e.g., Novartis' Concept1 inhaler (R=0.07 cm H₂O^{0.5}/L/min)—designated as C1). As a result, values of dₐ needed to achieve low deposition in the oropharynx can be larger for a high resistance inhaler such as the Simoon device. The Simoon inhaler is described, for example, in U.S. Pat. No. 8,573,197, and the Concept1 inhaler is described for example in U.S. Pat. No. 8,479,730.

In some embodiments of the invention, an ensemble of particles and particle agglomerates of the dry powder formulation present in the delivered dose suitably have a mass median aerodynamic diameter (MMAD) of between 1.0 and 3.0 μm, for example of between 1.5 and 2.0 μm. MMAD values around 2.0 μm are particularly preferred, as this provides low values of the inertial parameter, while limiting the fraction of particles that are exhaled even if subjects do not perform a suitable breath-hold.

Based on equation 1, decreases in dₐ can be achieved via corresponding decreases in d_{g}. While this is true for a single particle, this relationship is far more complicated for ensembles of particles, due to the formation of particle agglomerates. Hydrodynamic forces in the form of drag and lift forces are often used to fluidize and disperse particle agglomerates in dry powder inhalers. These forces decrease as the geometric size of the particles is decreased and are proportional to d_{g}². As a result there is a practical lower limit for d_{g}, below which decreases in geometric size result in increases in aerodynamic diameter, as particle agglomerates are poorly dispersed.

In some embodiments the primary particles of the dry powder formulation of the present invention suitably have a geometric size, expressed as a mass median diameter (×50) of between 0.8 and 2.5 μm, for example of between 0.9 and 2.4 μm, or 1.0 and 2.3 μm, or 1.2 and 2.2 μm.

In some embodiments the primary particles of the dry powder formulation of the present invention suitably have a geometric size, expressed as ×90 of between 2.0 μm and 4.0 μm, for example between 2.2 μm and 3.9 μm, or 2.3 μm and 3.7 μm, or 2.4 μm and 3.6 μm, or 2.5 μm and 3.5 μm.

While the median geometric size of the primary particles cannot go below about 1 μm, the aerodynamic size of the primary particles (Dₐ) must be significantly less than 1.0 μm in order for agglomerates of primary particles to remain respirable. This may be achieved by lowering the tapped density of the bulk powder. In some embodiments, having nanosized primary particles from an aerodynamic perspective is important to achieving a high TLD, as agglomerates of these primary particles must also be respirable with an MMAD of about 2 μm.

In some embodiments the primary particles of the dry powder formulation of the present invention suitably have a tapped density (ρ_{tapped}) of between 0.03 and 0.40 g/cm³, for example of between 0.07 and 0.30 g/cm³.

In some embodiments the primary particles of the dry powder formulation of the present invention suitably have a Dₐ of between 0.1 and 1.0 μm, for example between 0.5 and 0.8 μm.

Embodiments of the present invention comprise engineered particles comprising a porous, corrugated, or rugous surface. Such particles exhibit reduced interparticle cohesive forces compared to micronized drug crystals of a comparable primary particle size. This leads to improvements in powder fluidization and dispersibility relative to ordered or interactive mixtures of micronized drug and coarse lactose. In some embodiments, providing corrugated particles with a high degree of rugosity is important to achieve TLD>90%.

Embodiments of the present invention provide particles of a dry powder formulation of the invention which suitably have a rugosity of greater than 1, and below 30, for example from 1.5 to 20, 3 to 15, or 5 to 10.

For some active pharmaceutical ingredients a rugous surface is achieved via spray-drying of the neat active agent or drug. Such is often the case where the active agent or drug comprises a peptide or small protein (e.g., insulin). In some embodiments, peptides or small proteins comprise those having a molecular weight of between about 6000 and 20,000 Daltons. In such a case, the formulation may comprise neat drug, that is approximately 100% w/w of active agent or drug.

Embodiments of the present invention comprise formulations of drug and buffer, such as 95% or 96% or 97% or 98% or 99% or greater drug and the remainder, buffer. Embodiments of the present invention may comprise 70% to 99% w/w of drug or active agent, such as 70% to 95%.

For larger sized proteins (e.g., monoclonal antibodies and/or certain fragments thereof), the spray-dried particles do not naturally adopt a corrugated morphology. Under these circumstances, a platform core-shell dry powder formulation is preferred. Such a formulation comprises a shell-forming excipient to engender a corrugated morphology, and optionally additional buffer and/or glass-forming excipients to physically and chemically stabilize the amorphous glass.

Embodiments of core-shell dry powder formulations of the present invention may comprise 0.1 to 70% w/w of active agent, or 0.1 to 50% w/w of active ingredient(s), or 0.1% to 30% w/w of active ingredient(s).

In one or more embodiments of the dry powder formulation of the present invention, the formulation may additionally include excipients to further enhance the stability or biocompatibility of the formulation. For example, various salts, buffers, antioxidants, shell-forming excipients, and glass forming excipients are contemplated.

In some versions, the invention provides a system and method for both aerosolizing a powder pharmaceutical formulation comprising an active agent, and for for delivering the pharmaceutical formulation to the respiratory tract of the user, and in particular to the lungs of the user.

In some embodiments, the invention provides a formulation and process optimized for bypassing deposition in the upper respiratory tract, thereby minimizing tolerability or safety issues associated with drug deposition in the mouth and throat.

In some embodiments, the invention provides a formulation and process optimized for delivery of high doses (>10 mg) of a powder pharmaceutical formulation to the lungs.

In some embodiments, the invention provides a formulation and process optimized for systemic delivery of a powder pharmaceutical formulation comprising macromolecules via the respiratory tract.

Embodiments of present invention comprise spray-dried powders comprising neat APIs wherein particles of the powder have sufficient rugosity to result in a TLD of greater than 80% or 85% or 90% or 92%, or 95% or more of the nominal dose. Embodiments of the present invention include powders comprising more complex formulations comprising APIs and excipients that are utilized to stabilize the amorphous solid against both physical and chemical degradation, wherein the powder results in a TLD of greater than 80% or 85% or 90% or 92%, or 95% or more of the nominal dose.

### The Active Agent

Embodiments of the present invention are especially suited for the systemic delivery of various active agents including: peptides and proteins such as insulin and other hormones, active agents for targeting the central nervous system, and active agents for targeting the cardiovascular system. Embodiments of the present invention are also well suited for delivery to the peripheral airways for the treatment of respiratory diseases. Due to the high efficiency of delivery, the technology embodiments of the present invention are well suited for the delivery of active agents with a lung dose greater than 10 mg, including anti-infectives and antibodies.

The active agent described herein includes an agent, drug, compound, composition of matter or mixture thereof which provides some pharmacologic, often beneficial, effect. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the histamine system, and the central nervous system. Suitable active agents may be selected from, for example, hypnotics and sedatives, tranquilizers, respiratory drugs, drugs and biologics for treating asthma and COPD, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, antibodies, antibody fragments, proteins, polysaccharides, steroids, proteins capable of eliciting physiological effects, nucleotides, oligonucleotides, polynucleotides, fats, electrolytes, and the like.

In embodiments of the invention, the active agent may include or comprise any active pharmaceutical ingredient that is useful for treating inflammatory or obstructive airways diseases, such as asthma and/or COPD. Suitable active ingredients include long acting beta 2 agonist, such as salmeterol, formoterol, indacaterol and salts thereof, muscarinic antagonists, such as tiotropium and glycopyrronium and salts thereof, and corticosteroids including budesonide, ciclesonide, fluticasone, mometasone and salts thereof. Suitable combinations include (formoterol fumarate and budesonide), (salmeterol xinafoate and fluticasone propionate), (salmeterol xinofoate and tiotropium bromide), (indacaterol maleate and glycopyrronium bromide), and (indacaterol and mometasone).

In embodiments of the invention, the active agent may include or comprise antibodies, antibody fragments, nanobodies and other antibody formats which may be used for the treatment of allergic asthma including: anti-IgE, anti-TSLP, anti-IL-5, anti-IL-4, anti-IL-13, anti-CCR3, anti-CCR-4, anti-OX4OL.

In embodiments of the invention, the active agent may include or comprise proteins and peptides, such as insulin and other hormones; polysaccharides, such as heparin; nucleic acids, such as plasmids, oligonucleotides, aptamers, antisense, or ssRNA, dsRNA, siRNA; lipids and lipopolysaccharides; and organic molecules having biologic activity such as antibiotics, anti-inflammatories, cytotoxic agents, antivirals, vaso- and neuroactive agents.

Peptides and proteins may include hormones and cytokines such as insulin, relaxin, follicle stimulating hormone, parathyroid hormone, vasointestinal peptide, Agouti peptide, hemagglutinin peptide, interleukin-12, calcitonin, ostabolin C, leuprolide, elcitonin, oxytocin, carbetocin, somatostatin, pramlintide, amylin, glucagon, C-peptide, glucagon-like peptide 1 (GLP-1), erythropoietin, interferon α, interferon β, interleukin-1-r, interleukin-2, interleukin-13 receptor antagonist, interleukin-4 receptor antagonist, IL-4/IL-13 inhibitors, GM-CSF, Factor VIII, Factor IX, cyclosporine, a-1-proteinase inhibitor, human serum albumin, DNase, bikunin.

In embodiments of the invention, the active agent comprises an antimigraine drug including rizatriptan, zolmitriptan, sumatriptan, frovatriptan or naratriptan, loxapine, amoxapine, lidocaine, verapamil, diltiazem, isometheptene, lisuride; or anti-histamine drug including: brompheniramine, carbinoxamine, chlorpheniramine, azatadine, clemastine, cyproheptadine, loratadine, pyrilamine, hydroxyzine, promethazine, diphenhydramine; or anti-psychotic including olanzapine, trifluoperazine, haloperidol, loxapine, risperidone, clozapine, quetiapine, promazine, thiothixene, chlorpromazine, droperidol, prochlorperazine and fluphenazine; or sedatives and hypnotics including: zaleplon, zolpidem, zopiclone; or muscle relaxants including: chlorzoxazone, carisoprodol, cyclobenzaprine; or stimulants including: ephedrine, fenfluramine; or antidepressants including: nefazodone, perphenazine, trazodone, trimipramine, venlafaxine, tranylcypromine, citalopram, fluoxetine, fluvoxamine, mirtazepine, paroxetine, sertraline, amoxapine, clomipramine, doxepin, imipramine, maprotiline, nortriptyline, valproic acid, protriptyline, bupropion; or analgesics including: acetaminophen, orphenadrine and tramadol; or antiemetics including: dolasetron, granisetron and metoclopramide; or opiods including: naltrexone, buprenorphine, nalbuphine, naloxone, butorphanol, hydromorphone, oxycodone, methadone, remifentanil, or sufentanil; or antiParkinson compounds including: benzotropine, amantadine, pergolide, deprenyl, ropinerole; or antiarrhythmic compounds including: quinidine, procainamide, and disopyramide, lidocaine, tocamide, phenyloin, moricizine, and mexiletine, flecamide, propafenone, and moricizine, propranolol, acebutolol, soltalol, esmolol, timolol, metoprolol, and atenolol, amiodarone, sotalol, bretylium, ibutilide, E-4031 (methanesulfonamide), vernakalant, and dofetilide, bepridil, nitrendipine, amlodipine, isradipine, nifedipine, nicardipine, verapamil, and diltiazem, digoxin and adenosine. Of course, active agents may comprise pharmaceutically and formulation appropriate combinations of the foregoing.

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 100% by weight active agent, typically from about 2% to about 95% by weight active agent, and more typically from about 5% to 85% by weight active agent, and will also depend upon the relative amounts of additives contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

In some embodiments, pharmaceutical compositions are provided comprising at least one TSLP-binding molecule (e.g. antibody fragment) and at least one pharmaceutically acceptable excipient. In some embodiments, an excipient:TSLP-binding molecule mass ratio is greater than 0.5. In some embodiments, the TSLP-binding molecule is about 40-50% (w/w) of the pharmaceutical composition. In some embodiments, the pharmaceutical compositions comprise a shell-forming agent, such as trileucine or leucine. In some embodiments, the trileucine or leucine is about 10-75% (w/w) of the composition. In some embodiments, trileucine is about 10-30% (w/w) of the composition. In some embodiment, leucine is about 50-75% (w/w) of the composition. In some embodiments, the pharmaceutical compositions comprise at least one glass-forming excipient, such as trehalose, mannitol, sucrose, or sodium citrate. In some embodiments, at least one glass-forming excipient is trehalose or a mixture of trehalose and mannitol. In some embodiments, the glass-forming excipient is about 15-35% (w/w) of the composition. In some embodiments, the pharmaceutical compositions comprise a buffer, such as a histidine, glycine, acetate, or phosphate buffer. In some embodiments, the buffer is about 5-13% of the composition.

In some embodiments the TSLP-binding molecule comprises a monoclonal antibody or antibody fragments thereof such as Fab, Fab′, F(ab′)2, scFv, minibody, or diabody, that specifically bind human thymic stromal lymphopoietin (TSLP).

### Core Shell Particles

In some embodiments, the dry powder formulation of the present invention comprises core-shell particles comprising: a shell-forming excipient, and a core comprising the API, glass-forming excipients, and a buffer, sometimes also referred to herein as the platform formulation, or shell core platform formulation.

In some embodiments, the dry powder formulation of the present invention contains a pharmaceutically acceptable hydrophobic shell-forming excipient. The hydrophobic shell-forming excipient may take various forms that will depend at least to some extent on the composition and intended use of the dry powder formulation. Suitable pharmaceutically acceptable hydrophobic excipients may, in general, be selected from the group consisting of long-chain phospholipids, hydrophobic amino acids and peptides, and long chain fatty acid soaps.

in embodiments of the present invention, shell-forming excipients include: dipalmitoylphosphatidylcholine (DPPC), distearoylphosphatidylcholine (DSPC), magnesium stearate, leucine, dileucine, trileucine and combinations thereof. Particularly preferred are leucine and/or trileucine.

The evaporation of the volatile liquid components in an atomized droplet during spray-drying can be described as a coupled heat and mass transport problem. The difference between the vapor pressure of the liquids and their partial pressure in the gas phase is the driving force for the drying process. Two characteristic times are critical, determining the morphology of the spray-dried particles and the distribution of solid materials within the dried particles. The first is the time required for a droplet to dry, τ_{d}, and the second is the time required for materials in the atomized droplet to diffuse from the edge of the droplet to its center, R²/D. Here, R is the radius of the atomized droplet and D is the diffusion coefficient of the solutes or emulsion droplets present in the feedstock. The ratio of these two characteristic times defines the Peclet number,

$$Pe = \frac{{R}^{2}}{{τ}_{d} D} ,$$

a dimensionless mass transport number that characterizes the relative importance of the diffusion and convection processes. In the limit where drying of atomized droplets is sufficiently slow (Pe<<1), the components have an adequate time to redistribute by diffusion throughout the evaporating droplet. The end result is relatively dense particles (particle density true density of the components) with a homogenous composition. By contrast, if the drying of the atomized droplets is rapid (Pe>>1), components have insufficient time to diffuse from the surface to the center of the droplet and instead accumulate near the drying front of the atomized droplet. In such a case, low density particles with a core/shell distribution of components may occur.

In the context of the present invention, Pe depends on both formulation composition as well as the process, wherein material properties affect diffusion rates and process parameters affect drying rate. Although the concept of Peclet number is useful in engineered particle design, one must recognize that it is a simplification given that the composition of the liquid droplet, and therefore, the Pe of each component changes over the drying process. The hydrophobic shell-forming excipients disclosed herein precipitate early in the drying event, forming a shell on the drying droplet. After precipitation occurs, the diffusion of the excipient is no longer determined by its molecular diffusivity, but by the lower mobility of the phase-separated domains.

In some embodiments, the invention provides a formulation and process wherein the surface of the spray-dried particles is comprised primarily of the shell-forming excipient. Surface concentrations may be greater than 70%, such as greater than 75% or 80% or 85%. In some embodiments the surface is comprised of greater than 90% shell-forming excipient, or greater than 95% or 98% or 99% hydrophobic excipient. For potent APIs it is not uncommon for the surface to be comprised of more than 95% shell-forming excipient. The above-recited percentages refer to mass fraction of excipient on the particle surface.

In certain preferred embodiments the shell-forming excipient comprises greater than 70% of the particle surface (mass fraction) as measured by Electron Spectroscopy for Chemical Analysis (ESCA, also known as X-ray photoelectron spectroscopy or XPS), preferably greater than 90% or 95%.

In some embodiments the shell-forming excipient facilitates development of a rugous particle morphology. This means the particle morphology is porous, wrinkled, corrugated or creased rather than smooth. Hence the exterior surface of the inhalable particles (whether with or without drug or active agent) are at least in part rugous. This rugosity is useful for providing dose consistency and drug targeting by improving powder fluidization and dispersibility. Increases in particle rugosity result in decreases in inter-particle cohesive forces as a result of an inability of the particles to approach to within van der Waals contact. The decreases in cohesive forces are sufficient to dramatically improve powder fluidization and dispersion in ensembles of rugous particles.

If present, content of the shell-forming excipient generally ranges from about 15 to 50% w/w of the total particle mass (e.g. active agent, or active agent plus excipient). For embodiments comprising trileucine, a minimum of about 15% is preferred in the formulation to provide acceptable performance as a shell-former. For embodiments comprising leucine, the minimum preferred content is higher, about 30%.

The use of hydrophobic shell-forming excipients such as trileucine may be limited by their solubility in the liquid feedstock. Typically, the content of trileucine in an engineered powder is less than 30% w/w, more often on the order of 10% w/w to 20% w/w. Owing to its limited solubility in water and its surface activity, trileucine is an excellent shell former. Leucine may also be used as a shell forming excipient and embodiments of the invention may comprise particles which achieve leucine concentrations of up to about 50%. Fatty acid soaps (e.g., magnesium stearate) behave similarly to leucine and trileucine, and are thus suitable surface modifiers.

Due to the short timescale of the drying event, APIs that are dissolved in the feedstock will generally be present as amorphous solids in the spray-dried drug product.

The molecular mobility of an amorphous solid is significant when compared to that of its crystalline counterpart. Molecular mobility comprises long-range motions related to molecular diffusion as well as local motions such as bond rotations. The central principle in solid-state stabilization of amorphous materials is that molecular mobility leads to undesirable physical and chemical changes. Therefore, formulation strategies for amorphous materials usually focus on suppression of molecular mobility.

The existence of a relationship between molecular mobility and instability is well known to the art. However, to be a useful concept in particle engineering, molecular mobility must be carefully defined and understood in terms of the types of motions present. Long-range molecular motions arise from structural relaxation, known as α-relaxation. The timescale for such motions increases markedly as temperature decreases below the glass transition temperature (T_{g}), or conversely, as the T_{g} is raised at a fixed observation temperature. Because stabilization of a molecule in a glass limits its long-range molecular mobility, this has become the most common formulation strategy for solid-state stabilization of amorphous drugs.

When a glass-forming agent is needed, one or more considerations govern its selection. The primary role of a glass-forming excipient is to reduce the overall long-range molecular mobility of the drug. In practice, this is accomplished by raising the glass transition temperature of the amorphous phase that contains the drug. While excipients with high T_{g} values are generally desirable, even an excipient with a moderate T_{g} could be suitable for some formulations (e.g., drugs with a moderate T_{g} or if the drug concentration in the formulation is low). To guide the formulator, it is worthwhile to highlight the properties of an ideal glass-former: a biocompatible material with a high glass transition temperature that is miscible with the drug, forming a single amorphous phase that is only weakly plasticized by water.

Glass-forming excipients that suppress long-range molecular mobility, that is those which impart alpha relaxation, include carbohydrates, amino acids, and buffers. Particularly preferred glass-forming excipients include: sucrose, trehalose, and sodium citrate, with trehalose contemplated in embodiments of the present invention comprising a core-shell formulation and process.

The importance of other types of molecular motions has become increasingly recognized in the pharmaceutical literature. The nomenclature (α, β, etc.) used to designate the types of molecular motions originates from broadband dielectric spectroscopy. Dielectric relaxation spectra are conventionally plotted on a frequency scale. When these spectra are interpreted, the dielectric loss peaks at the lowest frequencies are designated as α motions, the higher frequency motions as β motions, then γ, and so forth. Thus, β and other motions that occur at higher frequencies are referred to as “fast” or secondary motions (and, in some cases, Johari-Goldstein relaxations). Although these secondary relaxations are often ascribed to intramolecular motions of different molecular moieties (e.g., side chains on a protein), they exist even for rigid molecules. In a simplistic physical picture, the β motions are sometimes described as random “cage rattling” of a species trapped among its nearest neighbors. At some point, the local motions of the nearest neighbors provide sufficient free volume to enable a diffusive jump of the trapped species. This is an α motion. Thus, the β motions can lead to a motions.

Secondary motions (β motions) are an area of active research. And, although much of the literature involves lyophilized or melt-quenched glasses, the principles are also relevant to amorphous, engineered particles for inhalation (e.g., powders manufactured using spray-drying or certain other bottom-up processes). Crystallization of small molecules near T_{g} has been suspected to arise from β motions. Protein formulators have recognized the importance of controlling these β motions. Suppression of β motions in amorphous formulations is typically done with small, organic excipients, such as glycerol, mannitol, sorbitol, and dimethylsulfoxide. Although these are the most frequently reported excipients to suppress β motions, other low MW organic molecules could serve this purpose (e.g., buffer salts or counterions). These excipients are hypothesized to suppress motions of high-mobility domains by raising the local viscosity. To the reader familiar with the vast literature on glassy stabilization, the use of such excipients might seem counterintuitive. These and most other low molecular weight materials have low T_{g} values and will reduce the T_{g} of a formulation, a phenomenon known as plasticization. However, these excipients can also diminish β motions. Thus, they are referred to as antiplasticizers or sometimes as plasticizers, depending on the point of reference; while they plasticize the a motions, they antiplasticize the β motions. Note that this terminology is a potential source of confusion in the literature; the designation of a material as a plasticizer or an antiplasticizer depends on whether one's point of reference is the α or the secondary (β) motions.

Because solid-state stabilization of proteins requires formulation of a glassy matrix, the contributions of α and β motions are of particular interest. Although the literature has numerous references of using glass-forming agents to stabilize proteins, until recently, there have been few specific references to the influence of these agents on local motions. Although the glass transition temperatures of proteins are difficult to measure, most data suggest that T_{g}>150° C. Thus, the excipients (e.g., disaccharides such as sucrose or trehalose) most commonly used to stabilize proteins will also plasticize the α motions in the protein (and antiplasticize secondary motions). Recent work has demonstrated that β motions largely govern the stability of proteins in sugar glasses. Thus, disaccharides antiplasticize β motions in protein formulations. Accordingly, in some embodiments comprising proteins as active agents, disaccharides are preferred excipients.

Embodiments of formulations of the present invention may comprise glass-forming excipients with a high glass transition temperature, for example greater than about 80° C. Embodiments of the present invention may comprise glass forming agents such as sucrose, trehalose, mannitol, fumaryl diketopiperazine, sodium citrate, and combinations thereof. Embodiments of formulations of the present invention may comprise glass-forming excipients with a moderate glass transition temperature, for example between about 50° C. and 80° C. It should be noted that the glass transition temperature of the excipient alone is secondary to the glass transition temperature of the excipient together with the target formulation. Thus, glass forming excipients are selected (either singly or in combination) to achieve the target glass transition temperature of the formulation.

In some embodiments, dry powder formulations of the present invention are prepared by spray drying a solution comprising API and glass forming excipients selected from those which are known to afford alpha relaxation (an alpha glass-former) and those which are known to afford beta relaxation (a beta-glass-former). By adjusting alpha and beta relaxations, the desired inhalation properties may be more readily obtained. This may be done for example by utilizing combinations of trehalose and mannitol.

The amount of glass former required to achieve suppress molecular mobility and achieve physical and chemical stability will be dependent on the nature of the active agent. For some embodiments with spray-dried proteins, the molar ratio of glass former to protein may be in the range from 300 to 900. For small molecules, the required amount of glass former will depend on the T_{g} of the active agent.

### Buffers/Optional Ingredients

Buffers are well known for pH control, both as a means to deliver a drug at a physiologically compatible pH (i.e., to improve tolerability), as well as to provide solution conditions favorable for chemical stability of a drug. In embodiments of formulations and processes of the present invention, the pH milieu of a drug (that is the pH in the matrix surrounding the drug, and to a certain extent, the pH of the drug particle itself) can be controlled by co-formulating the drug and buffer together in the same particle.

Buffers or pH modifiers, such as histidine or phosphate, are commonly used in lyophilized or spray-dried formulations to control solution- and solid-state chemical degradation of proteins. Glycine may be used to control pH to solubilize proteins (such as insulin) in a spray-dried feedstock, to control pH to ensure room-temperature stability in the solid state, and to provide a powder at a near-neutral pH to help ensure tolerability. Preferred buffers include: histidine, glycine, acetate, and phosphate. In some embodiments, histidine and/or histidine HCL can additionally or alternatively serve as a glass forming excipient.

Optional excipients include salts (e.g., sodium chloride, calcium chloride, sodium citrate), antioxidants (e.g., methionine), excipients to reduce protein aggregation in solution (e.g., arginine), taste-masking agents, and agents designed to improve the absorption of macromolecules into the systemic circulation (e.g., fumaryl diketopiperazine).

### Process

The present invention provides a process for preparing dry powder formulations for inhalation according to embodiments described herein. Exemplary formulations comprise spray-dried particles comprising at least one active agent, and having an in vitro total lung dose (TLD) of between 80 and 95% w/w, for example between 85 and 93% w/w of the nominal dose for an average adult subject.

The present invention provides a process for preparing dry powder formulations for inhalation comprising spray-dried particles, the formulation containing at least one active ingredient, and having an in vitro total lung dose (TLD) of between 90 and 100% w/w, for example between 90 and 95% w/w of the delivered dose for an average adult subject.

Embodiments of the present invention provide a process for preparing dry powder formulations for inhalation, comprising a formulation of spray-dried particles, the formulation containing at least one active ingredient that is suitable for treating obstructive or inflammatory airways diseases, particularly asthma and/or COPD.

Embodiments of the present invention provide a process for preparing dry powder formulations for inhalation, comprising a formulation of spray-dried particles, the formulation containing at least one active ingredient that is suitable for non-invasively treating diseases in the systemic circulation.

Spray drying confers advantages in producing engineered particles for inhalation such as the ability to rapidly produce a dry powder, and control of particle attributes including size, morphology, density, and surface composition. The drying process is very rapid (on the order of milliseconds). As a result most active ingredients which are dissolved in the liquid phase precipitate as amorphous solids, as they do not have sufficient time to crystallize.

Spray-drying comprises four unit operations: feedstock preparation, atomization of the feedstock to produce micron-sized droplets, drying of the droplets in a hot gas, and collection of the dried particles with a bag-house or cyclone separator.

Embodiments of the process of the present invention comprise three steps, however in some embodiments two or even all three of these steps can be carried out substantially simultaneously, so in practice the process can in fact be considered as a single step process. Solely for the purposes of describing the process of the present invention the three steps will be described separately, but such description is not intended to limit to a three step process.

In its fundamental form, a process of the present invention which yields dry powder particles comprises preparing a solution feedstock and removing solvent from the feedstock, such as by spray-drying, to provide the active dry powder particles.

In embodiments of the invention, the feedstock comprises at least one active dissolved in an aqueous-based liquid feedstock. In some embodiments, the feedstock comprises at least one active agent dissolved in an aqueous-based feedstock comprising an added co-solvent. Co-solvents may comprise ethanol, alkanols, ethers ketones and mixtures thereof. In general, such co-solvents are water miscible organic solvents.

The particle formation process is highly complex and dependent on the coupled interplay between process variables such as initial droplet size, feedstock concentration and evaporation rate, along with the formulation physicochemical properties such as solubility, surface tension, viscosity, and the solid mechanical properties of the forming particle shell.

For some embodiments of the present invention, it has been surprisingly discovered that the addition of small amounts of ethanol to the aqueous feedstock results in particles with a significantly lower particle density. This may be important for the achievement of high lung targeting, as it enables decreases in Dₐ. The addition of an ethanol co-solvent to an aqueous solution has a significant impact on the nature of the solvent system. Even at mass fractions as low as 5% w/w, the addition of ethanol results in significant increases in viscosity and decreases in surface tension, factors that will impact atomization, droplet evaporation, and particle corrugation. Moreover, the solubility of API in the feedstock may be decreased in the solvent mixture, resulting in precipitation of API earlier in the drying process.

In some embodiments, the feedstock comprises at least one active agent dissolved in an ethanol/water feedstock, wherein the fraction of ethanol is between 1% and 30% w/w, for example between 2% and 20% w/w, or 3% and 19% w/w, or 4% and 18% w/w, or 5% and 15% w/w or 6% and 12 w/w.

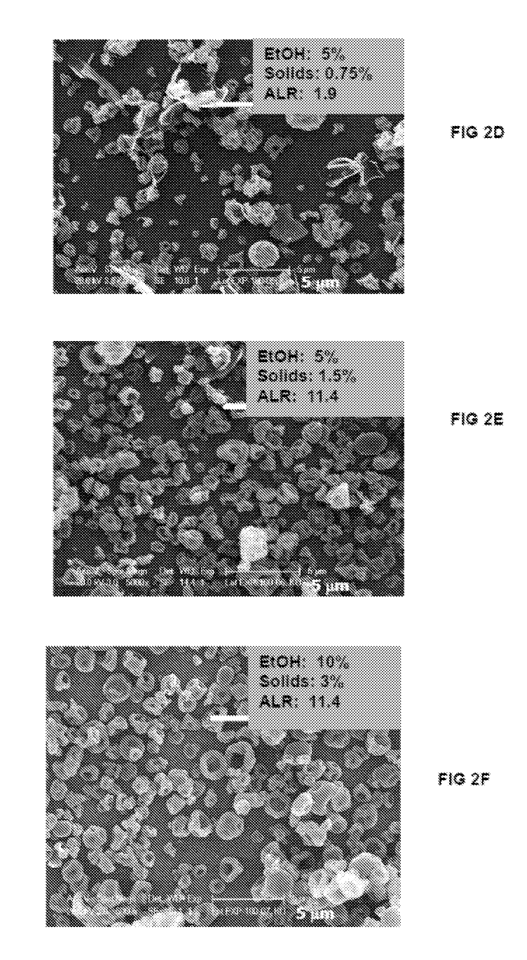

“Ethanol/solids ratio” refers to the ratio of the ethanol used as a co-solvent for the spray drying process to the total solids dissolved therein. Total solids includes API and any excipients. The ethanol/solids ratio has been found to correlate with the tapped or puck density of the spray-dried particles of the current invention (see FIG. 3). Generally favorable ethanol:solids ratios are between 1 and 20, for example between 2 and 15, or between 3 and 10. Typically, solids percentages within the solutions which are spray dried range from about 0.5 to about 2% w/w more typically 0.75 to 1.5% w/w.

For amorphous solids it is important to control the moisture content of the drug product. For drugs which are not hydrates, the moisture content in the powder is preferably less than 5%, more typically less than 3%, or even 2% w/w. Moisture content must be high enough, however, to ensure that the powder does not exhibit significant electrostatic attractive forces. The moisture content in the spray-dried powders may be determined by Karl Fischer titrimetry.

In some embodiments the feedstock is atomized with a twin fluid nozzle, such as that described in U.S. Pat. Nos. 8,524,279 and 8,936,813 (both to Snyder et al.). Significant broadening of the particle size distribution of the liquid droplets occurs above solids loading of about 1.5% w/w. The larger sized droplets in the tail of the distribution result in larger particles in the corresponding powder distribution. As a result, embodiments of a process of the present invention were in a twin fluid nozzle is employed generally restrict the solids loading to 1.5% w/w or less, such as 1.0% w/w, or 0.75% w/w.

In some embodiments, narrow droplet size distributions can be achieved with plane film atomizers as disclosed for example in U.S. Pat. Nos. 7,967,221 and 8,616,464 (both to Snyder et al.) at higher solids loadings. In some embodiments, the feedstock may be atomized at solids loading between 2% and 10% w/w, such as 3% and 5% w/w.

Any spray-drying step and/or all of the spray-drying steps may be carried out using conventional equipment used to prepare spray dried particles for use in pharmaceuticals that are administered by inhalation. Commercially available spray-dryers include those manufactured by Büchi Ltd. and Niro Corp.

In some embodiments, the feedstock is sprayed into a current of warm filtered air that evaporates the solvent and conveys the dried product to a collector. The spent air is then exhausted with the solvent. Operating conditions of the spray-dryer such as inlet and outlet temperature, feed rate, atomization pressure, flow rate of the drying air, and nozzle configuration can be adjusted in order to produce the required particle size, moisture content, and production yield of the resulting dry particles. The selection of appropriate apparatus and processing conditions are within the purview of a skilled artisan in view of the teachings herein and may be accomplished without undue experimentation. Exemplary settings for a NIRO® PSD-1® scale dryer are as follows: an air inlet temperature between about 80° C. and about 200° C., such as between 110° C. and 170° C.; an air outlet between about 40° C. to about 120° C., such as about 60° C. and 100° C.; a liquid feed rate between about 30 g/min to about 120 g/min, such as about 50 g/min to 100 g/min; total air flow of about 140 standard cubic feet per minute (scfm) to about 230 scfm, such as about 160 scfm to 210 scfm; and an atomization air flow rate between about 30 scfm and about 90 scfm, such as about 40 scfm to 80 scfm. The solids content in the spray-drying feedstock will typically be in the range from 0.5% weight/volume (w/v) (5 mg/ml) to 10% w/v (100 mg/ml), such as 1.0% w/v to 5.0% w/v. The settings will, of course, vary depending on the scale and type of equipment used, and the nature of the solvent system employed. In any event, the use of these and similar methods allow formation of particles with diameters appropriate for aerosol deposition into the lung.

As discussed previously for the particles comprising an amorphous API, the nature of the particle surface and morphology will be controlled by controlling the solubility and diffusivity of the components within the feedstock. Surface active hydrophobic excipients (e.g., trileucine, phospholipids, fatty acid soaps) may be concentrated at the interface, improving powder fluidization and dispersibility, while also driving increased surface roughness for the particles.

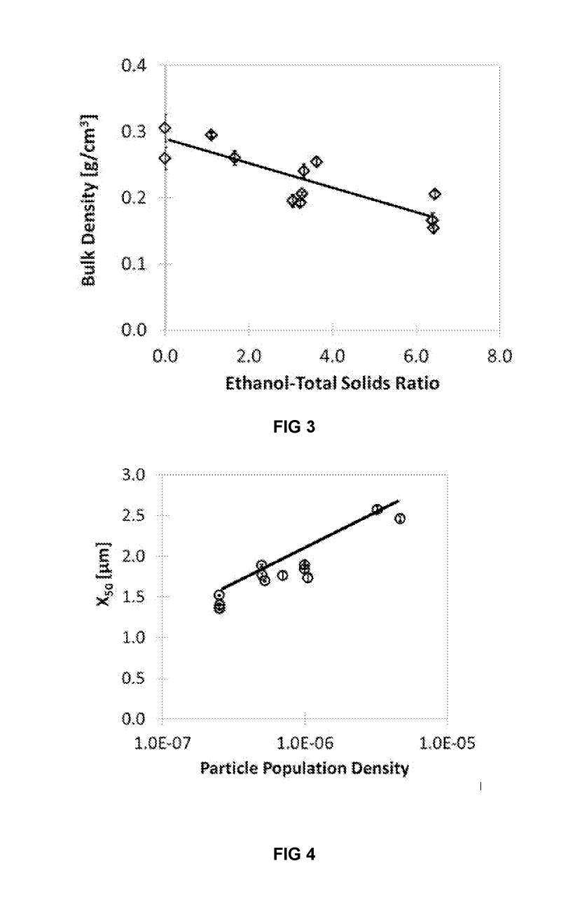

“Particle Population Density” (PPD) as used herein is a dimensionless number calculated from the product of the solids content and the atomizer liquid flow rate divided by the total dryer gas flow rate. The PPD has been observed to correlate with primary geometric particle size (see FIG. 4). More specifically, PPD is defined as the product of solids concentration in the feedstock and liquid feed rate divided by total air flow (atomizer air plus drying air). For a given system (considering spray drying equipment and formulation), the particle size, for example, the ×50 median size, of spray-dried powder is directly proportional to PPD. PPD is at least partially system dependent, therefore a given PPD number is not an universal value for all conditions.

In some embodiments a value of particle population density or PPD is between 0.01×10⁻⁶ and 1.0×10⁻⁶, such as between 0.03×10⁻⁶ and 0.2×10⁻⁶.

### Delivery System

The present invention also provides a delivery system, comprising an inhaler and a dry powder formulation of the invention.

In some embodiments, the present invention is directed to a delivery system, comprising a dry powder inhaler and a dry powder formulation for inhalation that comprises spray-dried particles that contain a therapeutically active ingredient, wherein the in vitro total lung dose is between 80% and 100% w/w of the nominal dose.

In some embodiments, the present invention is directed to a delivery system, comprising a dry powder inhaler and a dry powder formulation for inhalation that comprises spray-dried particles that contain a therapeutically active ingredient, wherein the in vitro total lung dose is between 90% and 100% w/w of the delivered dose.

### Inhalers

Suitable dry powder inhaler (DPIs) include unit dose inhalers, where the dry powder is stored in a capsule or blister, and the patient loads one or more of the capsules or blisters into the device prior to use. Alternatively, multi-dose dry powder inhalers are contemplated where the dose is pre-packaged in foil-foil blisters, for example in a cartridge, strip or wheel.

While any resistance of dry powder inhaler is contemplated, devices with a high device resistance (>0.13 cm H₂O^{0.5} L/min) may be preferred due to the lower flow rates that are achieved, thereby reducing the inertial parameter for a given sized particle.

Suitable dry powder inhaler (DPIs) include unit dose inhalers, where the dry powder is stored in a capsule or blister, and the patient loads one or more of the capsules or blisters into the device prior to use. Alternatively, multi-dose dry powder inhalers are contemplated where the dose is pre-packaged in foil-foil blisters, for example in a cartridge, strip or wheel.

Exemplary single dose dry powder inhalers include the AEROLIZER™ (Novartis, described in U.S. Pat. No. 3,991,761) and BREEZHALER™ (Novartis, described in U.S. Pat. No. 8,479,730 (Ziegler et al.). Other suitable single-dose inhalers include those described in U.S. Pat. Nos. 8,069,851 and 7,559,325.

Exemplary unit dose blister inhalers, which some patients find easier and more convenient to use to deliver medicaments requiring once daily administration, include the inhaler described by in U.S. Pat. No. 8,573,197 to Axford et al.

### Use in Therapy

Embodiments of the present invention provide a method for the treatment of an obstructive or inflammatory airways disease, especially asthma and chronic obstructive pulmonary disease, the method which comprises administering to a subject in need thereof an effective amount of the aforementioned dry powder formulation.

Embodiments of the present invention provide a method for the treatment of systemic diseases, the method which comprises administering to a subject in need thereof an effective amount of the aforementioned dry powder formulation.

### EXAMPLES

### Example 1—Preparation of Spray-Dried Formulations of Neat API

In Example 1, dry powder formulations of the invention containing neat recombinant human insulin were prepared by spray drying an aqueous-based feedstock containing ethanol as a co-solvent. Insulin is a small protein with a molecular weight of about 5,800 Da. The objective of this example was to produce a series of formulations with varying micromeritic properties (e.g., particle density and particle diameter) to optimize in vitro total lung deposition. Accordingly, particle properties were modulated by varying feedstock composition (i.e., total solids content, and ethanol-to-water ratio of the solution feedstock), and drying parameters (e.g., atomizer gas flow rate, liquid feed rate, air to liquid ratio (ALR) in atomizer, inlet temperature, and drying gas flow rate). The study used recombinant human insulin (P/N 10112053, Diabel GmbH & Co KGT in Frankfurt, Industriepark Höchst G680m Germany HMR4006). Feedstock solutions for spray drying were prepared by dissolving insulin powder in water or water-ethanol mixtures while mixing gently on a magnetic stir plate. The pH was lowered with hydrochloric acid (pH 3.0-3.25) to facilitate rapid dissolution of the drug substance, and then adjusted with sodium hydroxide to bring the final solution feedstock back to pH 7.5-7.9. Thus, although not quantitated, the formulation contained small amounts of sodium chloride.

This investigation used a Novartis Spray Dryer (NSD, Novartis Pharmaceuticals Corp, San Carlos, Calif.) a custom-built bench-scale spray dryer, that is similar in scale to the commercially available Büchi 191 mini spray dryer (BÜCHI Labortechnik, AG). The air-assisted atomizer nozzle is a modified version of Büchi 191 atomizer, designed to produce sprays with smaller and more uniform droplet size. The NSD dryer body and cyclone collector are fabricated out of stainless steel. The dryer body is insulated to improve temperature and relative humidity control within the process stream.

The compositions of the aqueous feedstocks and drying parameters for seven spray-dried formulations of neat insulin are presented in Table 1.

TABLE 1
Feedstock compositions and physicochemical
properties of neat insulin formulations

| | Ethanol | Atomizer | Liquid | | Inlet | Drying | Solids |
|---|---|---|---|---|---|---|---|
| | Fraction | Gas Flow | Feed Rate | ALR | Temp | Gas Flow | Content |
| Lot No. | (% w/w) | (L/min) | (mL/min) | (×10³ v/v) | (° C.) | (L/min) | (% w/w) |
| 100-01 | 5 | 15 | 8.0 | 1.9 | 115 | 700 | 5.0 |
| 100-02 | 5 | 15 | 4.0 | 3.8 | 110 | 500 | 5.0 |
| 100-03 | 0 | 26 | 2.3 | 11.4 | 103 | 560 | 0.75 |
| 100-04 | 5 | 26 | 2.3 | 11.4 | 103 | 560 | 0.75 |
| 100-05 | 5 | 15 | 8.0 | 1.9 | 115 | 700 | 0.75 |
| 100-06 | 5 | 26 | 2.3 | 11.4 | 103 | 560 | 1.5 |
| 100-07 | 10 | 26 | 2.3 | 11.4 | 103 | 560 | 3.0 |

### Example 2—Micromeritic Properties of Spray-Dried Formulations of Neat Insulin

The micromeritic properties of the formulations of Example 1 are presented in Table 2. The primary particle size distribution (PPSD) of inhaled insulin powder was measured with a Sympatec HELOS Type BF Model Laser Light Diffraction Analyzer (Sympatec GmbH, Germany), a RODOS-M (OASIS) dry powder disperser, and an ASPIROS powder dosing unit. The instrument evaluation mode was set to high resolution laser diffraction (HRLD), which returns size distributions based on Fraunhofer diffraction theory. Powder samples of 5-15 mg of powder were placed into a 1 mL vial and loaded into the ASPIROS dosing unit set at a speed of 25 mm·s⁻¹. The injector and primary pressure settings for the RODOS dry disperser were 4 mm and 4 bar, respectively. Measurements were performed using the R1 lens (R1: 0.1/0.18-35 μm). The RODOS settings were selected after verifying that they achieved essentially complete dispersion of the engineered powder down to the primary particles formed during the spray drying process. Three replicate measurements were performed for each powder formulation. Results are reported in terms of the volume median diameter, VMD or ×50 (mean of three replicates).

No direct measurement of particle density exists. For this Example, puck densities at a specified level of compression were measured as a surrogate for particle density. Bulk powder was compacted into a 0.0136 cubic centimeter cavity tool using vacuum suction at a pressure of 81 kPa. Excess powder was doctored off. The resulting powder puck was expelled from the cavity with a burst of compressed air at 5-10 psig, and the mass of powder determined on a Mettler Toledo AX206 balance (n=3-5 replicates). The resulting puck densities were lower than the corresponding particle densities, but the trends in values are expected to be similar.

Volume weighted median diameters (×50) for the seven spray-dried powders varied from 1.36 to 2.58 μm, while puck densities varied from 0.15 to 0.30 g/cm³.

The median aerodynamic diameter for the primary particles (Dₐ) was calculated based on the product of the ×50 multiplied by the square root of the puck density. Values of Dₐ varied from 0.58 to 1.41 μm.

TABLE 2
Micromeritic properties of spray-dried powders of neat insulin

| | Ethanol | Solids | | Puck | |
|---|---|---|---|---|---|
| | Fraction | Content | x50 | Density | Dₐ |
| Lot No. | (% w/w) | (% w/w) | (μm) | (g/cm³) | (μm) |
| 100-01 | 5 | 5.0 | 2.46 | 0.30 | 1.35 |
| 100-02 | 5 | 5.0 | 2.58 | 0.30 | 1.41 |
| 100-03 | 0 | 0.75 | 1.36 | 0.26 | 0.69 |
| 100-04 | 5 | 0.75 | 1.40 | 0.17 | 0.58 |
| 100-05 | 5 | 0.75 | 1.76 | 0.15 | 0.68 |
| 100-06 | 5 | 1.5 | 1.70 | 0.21 | 0.78 |
| 100-07 | 10 | 3.0 | 1.74 | 0.24 | 0.85 |

Particle morphology was assessed by scanning electron microscopy with a Philips XL 30 Environmental Scanning Electron Microscope (ESEM; Philips Electron Optics, US). A thin layer of bulk powder was placed on a 1 cm×1 cm silicon wafer disk (Omnisil, VWR IBSN3961559, US), and the sample was prepared for electron microscopy by sputter-coating a thin gold and palladium film (Denton, 21261 Cold Sputter/Etch and DTM-100, operated at <100 mTorr and 30-42 mA for 100-150 seconds). The coated samples were then loaded into the ESEM chamber and the filament current and accelerating voltage set to 1.6 A and 20 kV, respectively.

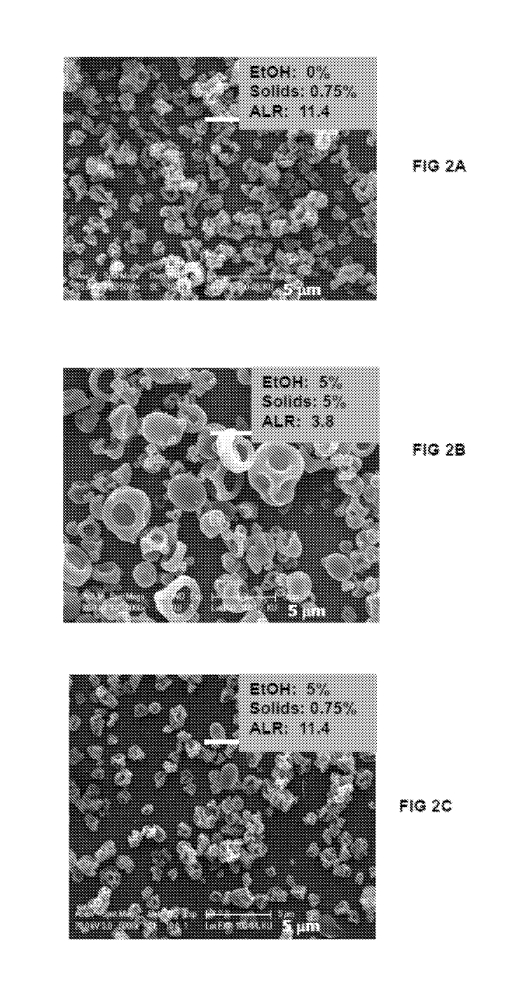

Scanning electron microscopy (SEM) images of the insulin inhalation powders are presented in FIG. 2. FIG. 2A represents a control powder produced by spray drying an aqueous feedstock with no added ethanol (100-03). The particles show a corrugated raisin-like morphology that is consistent with other formulations of spray dried proteins (e.g., Exubera®, Pfizer). The particles exhibit a relatively high puck density (0.26 g/cm³) and small primary particle size (1.36 μm). Formulation 100-04 (FIG. 2C) was manufactured with the same solids content, ALR, and drying conditions to the control powder, differing only in the composition of the liquid phase (5% w/w ethanol in the feedstock). The SEM image shows particle morphologies similar to those achieved for the control powder. Despite the lack of significant changes in ×50 or particle morphology, the puck density of the 100-04 powder was significantly lower (ρ_{puck}=0.17 g/cm³, ×50=1.40 μm). This is considered to result from the formation of particles with a decreased wall thickness.

Formulation 100-02 (FIG. 2B) was manufactured at a low ALR (3.8×10³ v/v) and high solids loading (5.0% w/v). The low ALR produces relatively large droplets, and the high solids content leads to precipitation of the particles earlier in the drying process. This results in larger-sized particles with a higher puck density (ρ_{puck}=0.30 g/cm³, ×50=2.58 μm). A mix of morphologies is observed with both corrugated particles and smooth oval shaped particles. In contrast, spray drying with a low ALR, low solids content (0.75%), and fast drying rates (Formulation 100-05) results is a complex mixture of particle morphologies (FIG. 2D). Interestingly, this formulation exhibits the lowest puck density of the formulations prepared (p_{puck}=0.15 g/cm³, ×50=1.76 μm). Compared to the control, the 100-05 formulation has a volume median diameter that is 0.4 μm larger. Formulations 100-06 (FIG. 2E) and 100-07 (FIG. 2F) were prepared at intermediate solids contents and exhibit physical properties intermediate to those discussed above. For example, formulations 100-04 and 100-06 differ only in the total solids, which increase from 0.75% to 1.5% w/v. This leads to an increase in ×50 from 1.40 to 1.70 μm and an increase in puck density from 0.17 to 0.21 g/cm³.

### Example 3—Aerosol Properties of Spray-Dried Formulations of Neat Insulin

Six of the spray dried insulin powders covering a wide range of puck densities (0.15-0.30 g/cm³) and volume median diameters (1.36-2.58 μm) were analyzed for in vitro aerosol performance.

In vitro dose delivery performance was investigated using two different dry powder inhalers (DPIs) that fluidize and disperse powder using different principles. The blister-based Simoon inhaler is a high resistance device (R about 0.19 cm H₂O^{0.5}/(L min⁻¹)) that utilizes airflow through an orifice to fluidize and de-agglomerate the powder. In contrast, the capsule-based T-326 inhaler is a low-medium resistance device (R about 0.08 cm H₂O^{0.5}/(L min⁻¹)), which relies on the mechanical motion associated with precession of the capsule to fluidize and disperse the bulk powder into a fine, respirable aerosol. Aerosol performance was evaluated using a standard square-wave flow profile generated with a timer-controlled vacuum source at pressure drops of 2, 4, and 6 kPa. This pressure drop range represents the range of inspiratory efforts achievable by most subjects, including both healthy volunteers and patients with obstructive lung disease.

Test attributes included the delivered dose (DD) measured gravimetrically for the neat insulin powders, the mass median aerodynamic diameter (MMAD) measured with a Next Generation Impactor, and an in vitro measure of total lung dose (TLD) determined with an idealized anatomical throat model. Numerous studies have demonstrated good in vitro-in vivo correlations (IVIVC) in total lung deposition for anatomical throats.

For delivered dose (DD) measurements, the aerosolized dose leaving the inhaler mouthpiece following aerosolization is deposited onto a filter (type A/E, Pall Corp, US) having a diameter of 47 mm (Simoon) or 81 mm (T-326). Data are presented as a percentage of the nominal dose (ND). Customized filter holders were designed for engineered particles, which allow for gravimetric analyses with both inhaler devices. The larger 81 mm diameter filter was used to minimize filter pressure drop for the T-326 device, which has a low flow resistance, and therefore a higher airflow during testing. A 2 L sampling volume was maintained for each dose actuation for DD. The results are presented in Table 3.

TABLE 3
Delivered dose of neat insulin formulations. Puck density (ρ) values are in units of g/cm³.

| | | Delivered Dose (% ND) Mean (SD) | | | | | |
|---|---|---|---|---|---|---|---|
| | ΔP/Q | 100-05 | 100-04 | 100-06 | 100-07 | 100-03 | 100-02 |
| Inhaler | (kPa/L/min) | ρ = 0.15 | ρ = 0.17 | ρ = 0.21 | ρ = 0.24 | ρ = 0.26 | ρ = 0.30 |
| Simoon | 2/23 | 96 (3) | 80 (35) | 88 (6) | 80 (7) | 61 (13) | 65 (6) |
| | 4/33 | 98 (6) | 96 (4) | 85 (10) | 78 (7) | 67 (17) | 75 (4) |
| | 6/41 | 98 (1) | 99 (2) | 93 (1) | 83 (2) | 81 (1) | 72 (4) |
| T-326 | 2/55 | — | 88 (4) | 85 (5) | — | 75 (5) | 84 (2) |
| | 4/78 | — | 90 (3) | 85 (1) | — | 80 (4) | 82 (2) |
| | 6/96 | — | 95 (4) | 86 (3) | — | 86 (4) | 81 (1) |

Significant improvements in DD are observed for both inhalers as the puck density of the powder is decreased. The decrease in DD is accompanied by a corresponding increase in the amount of powder retained in the blister or capsule. Delivered doses (ΔP=4 kPa) exceed 90% w/w when the puck density is in the range from 0.15 to 0.17 g/cm³.

In this regard, it has been surprisingly discovered that the addition of small amounts of ethanol to an aqueous-based feedstock enables significant reductions in puck density, while maintaining the corrugated particle morphology for fine particles less than 2 μm in size.

Modest differences in DD were observed for the various insulin formulations across the range of flow rates tested with the capsule-based T-326 Inhaler. For the Simoon Inhaler, increases in variability are noted at the low flow rate of 23 L/min. These differences are reflective of the different mechanisms of powder fluidization and dispersion in the two inhalers. Nonetheless, the DD is reasonably independent of flow rate across the range of pressure drops assessed.

In vitro estimates of TLD were obtained using an anatomical throat model, i.e., the Alberta Idealized Throat (AIT), which represents the mouth/throat geometry of an average human adult. The AIT was developed by Finlay and coworkers at the University of Alberta, Canada. For determination of in vitro TLD, the test inhaler was coupled to the inlet of the AIT, and the aerosol dose that bypasses impaction in the throat was collected downstream on a 76 mm diameter filter (A/E type, Pall Corp., US). A polysorbate (EMD Chemicals, Cat. #8170072, US) wetting agent (equal parts of Tween 20 and methanol, v/v) was used for coating the interior walls of the AIT to prevent particle re-entrainment. The results for the spray-dried insulin powders of Example 1 are presented in Table 4 (expressed as a percentage of the nominal dose), and Table 5 (expressed as a percentage of the delivered dose).

A high degree of lung targeting (TLD>90% w/w of the nominal dose) was observed for Lot 100-05 and Lot 100-04 (Table 4). Significant increases in TLD were observed with decreases in puck density (Table 4). The increase in the TLD appears to largely reflect the increase in DD described previously (Table 3). The low-density powders, exhibited an in vitro TLD for the T-326 and Simoon inhalers that are comparable to the DD, i.e., there was negligible deposition in the AIT (Table 5). In fact, TLD expressed as a percentage of the DD is high for all of the insulin powders, and throat deposition is extremely low. That is, the insulin particles bypass deposition in the throat and are effectively targeted to the lungs.

TABLE 4
In vitro total lung dose (TLD) of neat insulin formulations expressed as a percentage
of the nominal dose. Puck density (ρ) values are in units of g/cm³.

| | | Total Lung Dose (% ND) Mean (SD) | | | | | |
|---|---|---|---|---|---|---|---|
| | ΔP/Q | 100-05 | 100-04 | 100-06 | 100-07 | 100-03 | 100-02 |
| Inhaler | (kPa/L/min) | ρ = 0.15 | ρ = 0.17 | ρ = 0.21 | ρ = 0.24 | ρ = 0.26 | ρ = 0.30 |
| Simoon | 2/23 | 97 (5) | 82 (22) | 91 (3) | 79 (12) | 64 (18) | 63 (11) |
| | 4/33 | 96 (4) | 91 (4) | 83 (20) | 80 (7) | 70 (5) | 63 (7) |
| | 6/41 | 94 (9) | 94 (4) | 87 (4) | 82 (5) | 76 (4) | 69 (3) |
| T-326 | 2/55 | — | 90 (1) | 80 (4) | — | 74 (5) | 74 (3) |
| | 4/78 | — | 92 (4) | 83 (2) | — | 78 (2) | 65 (3) |
| | 6/96 | — | 91 (3) | 84 (3) | — | 79 (3) | 65 (3) |

TABLE 5
In vitro total lung dose (TLD) of neat insulin formulations expressed as a percentage
of the delivered dose. Puck density (ρ) values are in units of g/cm³.

| | | Mean Total Lung Dose (% DD) | | | | | |
|---|---|---|---|---|---|---|---|
| | ΔP/Q | 100-05 | 100-04 | 100-06 | 100-07 | 100-03 | 100-02 |
| Inhaler | (kPa/L/min) | ρ = 0.15 | ρ = 0.17 | ρ = 0.21 | ρ = 0.24 | ρ = 0.26 | ρ = 0.30 |
| Simoon | 2/23 | 101 | 103 | 103 | 101 | 105 | 97 |
| | 4/33 | 98 | 95 | 98 | 103 | 104 | 84 |
| | 6/41 | 96 | 95 | 94 | 99 | 94 | 96 |
| T-326 | 2/55 | — | 101 | 94 | — | 99 | 88 |
| | 4/78 | — | 102 | 98 | — | 93 | 79 |
| | 6/96 | — | 96 | 98 | — | 92 | 80 |

In vitro measurements of the mass median aerodynamic diameter (MMAD) were conducted for selected insulin powder formulations with a Next Generation Impactor (NGI) operated at a pressure drop of 4 kPa (i.e., 33 L/min for the Simoon device). Drug quantitation was performed gravimetrically. To enable gravimetric analysis, the gravimetric NGI cups were fitted with 55-mm diameter glass fiber filters (A/E type, Pall Corp, USA) and the pre-separator upper and lower compartments were coated with 1 ml and 2 ml, respectively, of a polysorbate wetting agent (equal parts of Tween 20 and methanol, v/v). The results are presented in Table 6.

TABLE 6
Mass median aerodynamic diameters of spray-
dried insulin powders delivered from the Simoon
Inhaler at a flow rate of 33 L/min (4 kPa)

| Lot # | 100-02 | 100-03 | 100-04 | 100-05 | 100-06 | 100-07 |
|---|---|---|---|---|---|---|
| MMAD (μm) | 3.14 | 1.90 | 1.78 | 2.02 | 2.00 | 2.26 |

With the exception of the larger sized particles obtained in the 100-02 lot, the remaining neat insulin powders have an MMAD of about 2 μm. It is worth noting that for lots 100-04 and 100-05, virtually the entire delivered dose is sampled on the impactor. That is, deposition of non-respirable particles in the pre-separator and USP throat is negligible. In contrast, current marketed products lose between 30% and 70% of particles in the pre-separator and USP throat, resulting in a significant underestimation of the true MMAD of the powder formulation.

Deposition in the mouth-throat is governed by inertial impaction, and as such depends critically on the inertial impaction parameter, dₐ²Q. The impact of variations in dₐ²Q on regional deposition in the respiratory tract for monodisperse liquid aerosols has been studied by workers in the art. Negligible deposition in the mouth-throat was observed for aerosols with dₐ²Q<120 μm² L/min. In embodiments and examples of the present invention, nearly 100% of the DD of lots 100-04 and 100-05 bypass deposition in the AIT, which means nearly 100% TLD. Utilizing the measured MMAD values and the test flow rate (33 L min⁻¹), the calculated median dₐ²Q values are 105 and 135 μm² L/min, respectively.

It has been previously demonstrated that a significant component of the variability in drug delivery to the lungs results from anatomical differences in a subject's mouth-throat. For current marketed portable inhalers where mean total lung deposition is on the order of 10-30%, the mean variability in TLD in vivo is approximately 30-50%. In the limit where particles are able to entirely bypass deposition in the mouth-throat, the variability in TLD would, by definition, be 0%. Hence, significant improvements in dose consistency are anticipated as the drug/device combinations are designed to minimize mouth-throat deposition. This may be especially important for drugs with a narrow therapeutic index like insulin, or drugs that elicit significant side-effects in the oropharynx, such as inhaled corticosteroids.

Finally, the small MMAD noted for these aerosols suggests that a significant fraction of the DD will be deposited in the peripheral airways. For proteins like insulin, it has been hypothesized that deposition in the lung periphery is critical for achieving effective absorption into the systemic circulation. Using a standard deposition model, an estimate of approximately 85% peripheral deposition is obtained for the polydisperse particle population in Lot 100-04. As a result, significant increases in systemic bioavailability are anticipated for inhaled macromolecules. This would be expected to significantly reduce the cost of goods for inhaled macromolecules. This may enable development of therapeutic proteins that might not otherwise be developable.

### Example 4: Design of Process for Insulin Inhalation Powders to Bypass Deposition in the Upper Respiratory Tract

Observed powder properties were quantitatively correlated to process and feedstock parameters. The results presented in FIG. 3 show that bulk density can be influenced by varying ethanol to total solids ratio in the solution feedstock. Low bulk densities were particularly favored for the spray-dried insulin powders when the total solids concentration was 0.75% w/w.

The diameter of a spray-dried particle is expected to scale with solids content and initial droplet diameter according to Equation 3:

$${d}_{g} = \sqrt[3]{\frac{{C}_{s} {ρ}_{s}}{{ρ}_{p}}} {d}_{d} \qquad ( Equation 3 )$$

where d_{d} is the initial diameter of the atomized droplet, Cₛ is total solids in the feedstock, ρₛ is the density of the feedstock solution, and ρₚ is the particle density. In the absence of experimental data on particle density and atomized droplet size, an empirically derived correlate for particle diameter has been proposed, i.e., the particle population density (PPD). The PPD is a dimensionless parameter defined in Equation 4:

$$PPD = \frac{{C}_{s} {Q}_{L}}{{Q}_{T}} \qquad ( Equation 4 )$$

where Q_{L} is the atomizer liquid flow-rate, and Q_{T} is the total dryer gas flow-rate. FIG. 4 is a plot showing the correlation between ×50 and PPD. The correlations based on the results from this co-solvent spray drying study with insulin suggest that feedstock and process parameters can be modulated to achieve a desired particle density and size to enable maximum targeting of aerosol to the lungs.

### Example 5: Preparation of Simple Spray-Dried Formulations of a Monoclonal Antibody Fragment

The monoclonal antibody fragment described herein comprises an anti-TSLP fragment and has a molecular weight of 46.6 kDa. Dry powder formulations are described for local lung delivery in the treatment of asthma. In this context, the use of the term “simple” refers to formulations of active and buffer only.

A series of simple antibody formulations comprising 89.5% active pharmaceutical ingredient and 10.5% histidine buffer were manufactured from feedstocks comprising various ethanol/water solvent compositions (Table 7). The ethanol content was varied between 5% and 20% w/w. The feedstocks were spray-dried on the NSD spray-dryer with an inlet temperature of 105° C., an outlet temperature of 70° C., a drying gas flow rate of 595 L/min, an atomizer gas flow rate of 20 L/min, a liquid feed rate of 8.0 mL/min, and an ALR of 2.5×10³ v/v. The solids content was fixed at 2% w/v.

TABLE 7
Impact of process parameters on micromeritic properties of simple
antibody formulations comprising 89.5% API in histidine buffer.

| | | | | | | | | Tapped |
|---|---|---|---|---|---|---|---|---|
| | API | Trileucine | Solids | EtOH | PPSD (μm) | | | Density |
| Lot # | (% w/w) | (% w/w) | (% w/v) | (% w/w) | ×10 | ×50 | ×90 | (g/cm³) |
| 761-22-07 | 89.5 | 0 | 2 | 0 | 0.55 | 1.34 | 3.24 | 0.347 |
| 761-02-09 | 89.5 | 0 | 2 | 5 | 0.66 | 1.93 | 5.64 | 0.178 |
| 761-02-06 | 89.5 | 0 | 2 | 10 | 0.73 | 2.48 | 7.19 | 0.142 |
| 761-02-07 | 89.5 | 0 | 2 | 20 | 0.69 | 1.94 | 6.04 | 0.135 |

### Example 6: Micromeritic Properties of Simple Spray-Dried Formulations of Antibody

The micromeritic properties of the spray-dried antibody formulations of Example 5 are presented in Table 7. All of the simple formulations comprising just API and buffer, produced particles with a smooth particle surface (i.e., no surface corrugation). The addition of small amounts of ethanol to the aqueous feedstock decreased the bulk and tapped density of the powders, in a manner similar to that observed for insulin formulations in Example 2. The particles were also significantly larger in terms of their primary particle size distribution (PPSD), than particles of the insulin formulations. However, as described herein, other particle characteristics, including rugosity and particle density, can be adjusted to balance a larger particle size distribution to result in the described high total lung dose of the present invention.

### Example 7: Aerosol Performance of Simple Spray-Dried Formulations of Antibody

The DD and TLD determined for the powders delineated in Example 6 are presented in Table 8. The primary particles had a calculated median aerodynamic diameter, Dₐ, between 0.71 and 0.93 μm (calculated from the tapped density and ×50 measurements using equation 1).

The Concept1 dry powder inhaler is a low resistance capsule-based device (R=0.07 cm H₂O)^{1/2}/(L/min)).

TABLE 8
Aerosol performance of simple antibody formulations.
Aerosol performance was assessed with the Concept1
Inhaler (20 mg fill mass) at a flow rate of 90 L/min
and a total volume of 2 L (n = 5).

| | Tapped | | Dₐ | | | |
|---|---|---|---|---|---|---|
| | Density | ×50 | (calc) | | DD | TLD |
| Lot # | (g/cm³) | (μm) | (μm) | Morphology | (% ND) | (% DD) |
| 761-22-07 | 0.347 | 1.34 | 0.79 | Smooth | 64.9 | 65.0 |
| 761-02-09 | 0.178 | 1.93 | 0.81 | Smooth | 77.0 | 57.1 |
| 761-02-06 | 0.142 | 2.48 | 0.93 | Smooth | 81.2 | 43.7 |
| 761-02-07 | 0.135 | 1.94 | 0.71 | Smooth | 74.3 | 57.7 |

It is clear from the data in Table 8 that, in some embodiments, decreasing density alone is insufficient to enable formation of particles that effectively bypass deposition in the mouth-throat. Therefore, in some embodiments, effectively bypassing mouth throat deposition (increasing TLD) may be attained by modifying particle morphology to increase surface rugosity (corrugation). In some embodiments, increasing TLD may be attained by decreases in primary particle size. In some embodiments increasing TLD may be attained by both increasing surface rugosity and decreasing primary particle size.

It is interesting to note that while peptides and small proteins naturally adopt a corrugated morphology in the absence of a shell-forming excipient, formulation of the antibody (and/or antibody fragment), in some embodiments, requires the addition of a shell-forming excipient to enable formation of corrugated particles. In this regard, the shell-forming excipient and addition of ethanol perform similar functions in modifying the wall thickness and density of the spray-dried particles. Hence the impact of addition of ethanol is smaller, in some embodiments, in the presence of a shell former.

### Example 8: Preparation and Micromeritic Properties of Platform Spray-Dried Formulations of Antibody

In this series of spray-dried powders, the spray-drying conditions were held constant, and the impact of the addition of a shell-forming excipient (i.e., trileucine, 0-15% w/w) was assessed for antibody formulations. These formulations also contain trehalose as a glass-former (about 29-44% w/w depending on trileucine content) and histidine buffer (5.9% w/w, pH 5.0).

Powders were spray-dried on the custom NSD spray dryer with an inlet temperature of 105° C., an outlet temperature of 70° C., a drying gas flow rate of 595 L/min, an atomizer gas flow rate of 25 L/min, a liquid feed rate of 10.0 mL/min, and an ALR of 2.5×10³ v/v. The solids content was held constant at 2% w/w. All of the powders had a corrugated morphology with the exception of lot 761-02-12, which was spray dried in the absence of a shell former and produced smooth particles similar to those observed in Example 7. Results are shown in Table 9.

TABLE 9
Impact of process parameters on micromeritic properties
of ‘platform’ antibody formulations comprising 50.0%
w/w API, 5.9% histidine buffer, trehalose and trileucine.

| | | | | | | | Tapped |
|---|---|---|---|---|---|---|---|
| | API | Trileucine | EtOH | PPSD (μm) | | | Density |
| Lot # | (% w/w) | (% w/w) | (% w/w) | ×10 | ×50 | ×90 | (g/cm³) |
| 728-06-04 | 50.0 | 10.0 | 0 | 0.55 | 2.28 | 5.14 | 0.366 |
| 728-06-02 | 50.0 | 15.0 | 0 | 0.64 | 2.06 | 4.83 | 0.197 |
| 761-02-12 | 50.0 | 0.0 | 10 | 0.48 | 1.60 | 4.87 | 0.158 |
| 761-22-06 | 50.0 | 5.0 | 10 | 0.50 | 1.63 | 3.85 | 0.268 |
| 761-02-11 | 50.0 | 10.0 | 10 | 0.63 | 2.25 | 5.75 | 0.176 |
| 761-02-10 | 50.0 | 15.0 | 10 | 0.67 | 2.30 | 5.27 | 0.112 |

### Example 9: Aerosol Performance of ‘Platform’ Spray-Dried Formulations of Antibody with Varying Trileucine Content

The DD and TLD described for the powders delineated in Example 8 are presented in Table 10.

TABLE 10
Impact of process parameters on micromeritic properties and aerosol
performance of platform antibody formulations. Aerosol performance
was assessed with the Concept1 Inhaler (20 mg fill mass) at a flow
rate of 90 L/min and a total volume of 2 L (n = 5).

| | | Tapped | | Dₐ | | | |
|---|---|---|---|---|---|---|---|
| | Ethanol/ | Density | ×50 | (calc) | | DD | TLD |
| Lot # | Solids | (g/cm³) | (μm) | (μm) | Morphology | (% ND) | (% DD) |
| 728-06-04 | 0 | 0.366 | 2.28 | 1.38 | Corrugated | 90.0 | 83.3 |
| 728-06-02 | 0 | 0.197 | 2.06 | 0.91 | Corrugated | 90.0 | 80.0 |
| 761-02-12 | 5 | 0.158 | 1.60 | 0.64 | Smooth | 69.0 | 66.2 |
| 761-22-06 | 5 | 0.268 | 1.63 | 0.84 | Corrugated | 89.2 | 79.1 |
| 761-02-11 | 5 | 0.176 | 2.25 | 0.94 | Corrugated | 92.3 | 84.8 |
| 761-02-10 | 5 | 0.112 | 2.30 | 0.77 | Corrugated | 93.1 | 83.0 |

Significant improvements in DD and TLD are observed for antibody formulations with a corrugated particle morphology. In embodiments of the invention, the desired corrugated morphology results from the presence of the shell-forming excipient trileucine on the particle surface.

In embodiments of the invention, physicochemical properties of the material on the surface of the particles influence particle morphology. For large proteins (such as certain proteins above 20,000 Daltons) a shell forming excipient such as trileucine is preferred to achieve the desired morphology. In embodiments of the invention particles forming the formulation and composition must have a corrugated morphology to reduce cohesive forces between particles, such that the size of the agglomerates is small enough that the agglomerates are respirable.

When ethanol is added, it lowers the particle density of (otherwise) corrugated particles by decreasing the wall thickness. This, in turn, lowers the tapped density enabling smaller primary particles in accord with desired aerodynamic properties. In some embodiments particles should have a lowered density, such that the primary particles, and the agglomerates, are respirable.

Significant reductions in tapped density are noted for paired formulations 728-06-04 and 761-02-11 and 728-06-02 and 761-02-10 when the ethanol content is increased from 0% to 10% w/w. For the specific formulations in this Example, addition of 10% ethanol alone did not afford the target improvement in aerosol performance over what is provided by the shell-forming excipient. The TLD is excellent (>80% of the DD), but remains below the desired target of 90% w/w of the DD, in large part because the particles are too large and dense. For the corrugated particles the calculated primary aerodynamic diameter, Dₐ, ranges from 0.77 to 1.38 μm.

### Example 10: Impact of Modified Process Parameters (Solids Content and Co-Solvent Addition) on Micromeritic Properties of Platform Antibody Formulations

Formulations comprising 50.0% w/w API, 5.9% w/w histidine buffer (pH 5.0), ˜14% w/w or 29% w/w trehalose and 15% w/w or 30% w/w trileucine. Powders were spray dried on a custom NSD spray dryer with an inlet temperature of 105° C., an outlet temperature of 70° C., a drying gas flow rate of 595 L/min, an atomizer gas flow rate of 30 L/min, a liquid feed rate of 4.0 mL/min, and an ALR of 7.5×10³ v/v. The solids content was reduced to 1% w/w. These modifications in the spray drying process were designed to reduce the primary particle size. Indeed significant reductions in the primary particle size distribution are observed.

TABLE 11
Impact of process parameters on micromeritic properties
of ‘platform’ antibody formulations comprising 50.0%
w/w API, 5.9% histidine buffer, trehalose and trileucine.

| | | | | | | | | Tapped |
|---|---|---|---|---|---|---|---|---|
| | API | Solids | Trileucine | EtOH | PPSD (μm) | | | Density |
| Lot # | (% w/w) | (% w/v) | (% w/w) | (% w/w) | ×10 | ×50 | ×90 | (g/cm³) |
| 761-22-01 | 50.0 | 1.0 | 15.0 | 5 | 0.39 | 1.33 | 2.59 | 0.282 |
| 761-22-02 | 50.0 | 1.0 | 15.0 | 10 | 0.51 | 1.31 | 2.59 | 0.232 |
| 761-22-03 | 50.0 | 1.0 | 15.0 | 20 | 0.53 | 1.36 | 2.94 | 0.151 |
| 761-02-04 | 50.0 | 1.0 | 15.0 | 30 | 0.55 | 1.44 | 3.15 | 0.162 |
| 761-22-05 | 50.0 | 1.0 | 30.0 | 20 | 0.64 | 1.58 | 2.94 | 0.122 |

### Example 11: Impact of Modified Process Parameters (Solids Content and Co-Solvent Addition) on Aerosol Performance of Platform Antibody Formulations

The impact of reductions in solids content and increases in ALR on aerosol performance of platform antibody formulations are presented in Table 12. Significant reductions in the median aerodynamic diameter of the primary particles are observed relative to the particles in Example 9. This translates into TLD values that are in some embodiments, between about 94% and 98% of the DD, i.e., within a desired, optimal or preferred target range of performance.

TABLE 12
Impact of process parameters on micromeritic properties and aerosol
performance of platform antibody formulations. Aerosol performance
was assessed with the Concept1 Inhaler (20 mg fill mass) at a flow
rate of 90 L/min and a total volume of 2 L (n = 5).

| | | Tapped | | | Dₐ | | |
|---|---|---|---|---|---|---|---|
| | Ethanol/ | Density | ×50 | | (calc) | DD | TL |
| Lot # | Solids | (g/cm³) | (μm) | Morphology | (μm) | (% ND) | (% DD) |
| 761-22-01 | 5 | 0.282 | 1.33 | Corrugated | 0.71 | 92.4 | 97.8 |
| 761-22-02 | 10 | 0.232 | 1.31 | Corrugated | 0.63 | 93.9 | 95.1 |
| 761-22-03 | 20 | 0.151 | 1.36 | Corrugated | 0.53 | 92.1 | 95.6 |
| 761-02-04 | 30 | 0.162 | 1.44 | Corrugated | 0.58 | 93.7 | 95.0 |
| 761-22-05 | 20 | 0.122 | 1.58 | Corrugated | 0.55 | 95.0 | 93.7 |

### Example 12: Preparation of Simple Spray-Dried Formulations of Serelaxin Under Various Process Conditions

Serelaxin (RLX030) is a peptide hormone of the relaxin-2 family with a molecular weight of about 6,000 Daltons.

Simple formulations comprising 80.0% w/w RLX030 and 20.0% w/w sodium acetate buffer were prepared at various contents of ethanol (0-20% w/w) in the liquid feedstock, various solids contents (0.75% to 1.5% w/w), and various ALR (2.5×10³ to 6.0×10³ v/v) in the twin fluid atomizer. Powders were spray-dried on a custom NSD spray drier. For lots 761-35-01 through 761-35-04 the inlet temperature was 105° C., the outlet temperature was 70° C., the drying gas flow rate was 595 L/min, the atomizer gas flow rate was 25 L/min, the liquid feed rate was 10.0 mL/min, and the ALR was 2.5×10³ v/v. For lots 761-35-05 through 761-35-09, the drying parameters were: inlet temperature of 105° C., outlet temperature of 70° C., a drying gas flow rate of 595 L/min, an atomizer gas flow rate of 30 L/min, a liquid feed rate of 5.0 mL/min, and an ALR of 6.0×10³ v/v.

### Example 13: Micromeritic Properties of Simple Spray-Dried Formulations of RLX030

The micromeritic properties for the lots produced in Example 12 are detailed in Table 13. Relative to the antibody formulations, the RLX030 formulations exhibit a smaller tapped density. As was observed with the insulin formulations, addition of small percentages of ethanol in the liquid feedstock lead to significant reductions in tapped density. Increases in ALR and reductions in solids content produce particles with a smaller primary particle size distribution (PPSD).

TABLE 13
Impact of variations in process parameters (e.g., ethanol
content, ALR, and solids content) on micromeritic properties
of simple RLX030 formulations comprising 80.0% w/w RLX030,
20.0% acetate buffer (N = 2, SD < 0.05 for all lots).

| | | | | | | | Tapped |
|---|---|---|---|---|---|---|---|
| | EtOH | Solids | | PPSD (μm) | | | Density |
| Lot # | (% w/w) | (% w/v) | ALR | ×10 | ×50 | ×90 | (g/cm³) |
| 761-35-01 | 0 | 1.5 | 2.5 | 0.75 | 2.16 | 4.18 | 0.16 |
| 761-35-02 | 5 | | | 0.79 | 2.22 | 4.99 | 0.08 |
| 761-35-03 | 10 | | | 0.77 | 2.19 | 4.91 | 0.07 |
| 761-35-04 | 20 | | | 0.75 | 2.01 | 4.24 | 0.09 |
| 761-35-05 | 0 | 1.0 | 6.0 | 0.74 | 1.74 | 3.20 | 0.18 |
| 761-35-06 | 5 | | | 0.72 | 1.69 | 3.44 | 0.10 |
| 761-35-07 | 10 | | | 0.67 | 1.58 | 3.14 | 0.11 |
| 761-35-08 | 20 | | | 0.65 | 1.49 | 2.92 | 0.11 |
| 761-35-09 | 5 | 0.75 | 6.0 | 0.68 | 1.58 | 3.06 | 0.11 |

### Example 14: Aerosol Performance of Simple Spray-Dried Formulations of RLX030 with Different Micromeritic Properties

The aerosol performance of the spray-dried RLX030 formulations detailed in Example 13 are detailed in Table 14. When manufactured with an ethanol co-solvent, the primary particles had a calculated median aerodynamic diameter of 0.5 to 0.6 μm. All of the lots produced with an ethanol co-solvent had a DD>90% of the ND, and a TLD>85% w/w of the DD, with most powders between 90% and 95% of the DD.

The lower the total solids concentration and the higher the ALR, the smaller the primary particle size. Addition of small amounts (5-20%) of ethanol help to reduce the density of the spray-dried particles. Earlier shell formation as well as ‘trapped vapour pressure’ inside the particles causes the creation of hollow particles with a decreased shell thickness and lower density. Addition of the specified amounts of ethanol, alone, help to improve aerosol performance. However, higher concentrations provided little additional benefit. Higher concentrations of ethanol or another co-solvent may be desired in some instances, to aid in the dissolution of the drug or active pharmaceutical ingredient. The desired solvent composition can easily be determined experimentally.

TABLE 14
Impact of process parameters on aerosol performance of simple RLX030
formulations comprising 80.0% w/w RLX030, 20.0% acetate buffer. Aerosol
performance was assessed with the Concept1 Inhaler (20 mg fill mass)
at a flow rate of 90 L/min and a total volume of 2 L (n = 5).

| | | Tapped | | Dₐ | | | |
|---|---|---|---|---|---|---|---|
| | Ethanol/ | Density | ×50 | (calc) | | DD | TLD |
| Lot # | Solids | (g/cm³) | (μm) | (μm) | Morphology | (% ND) | (% DD) |
| 761-35-01 | 0 | 0.16 | 2.16 | 0.86 | Corrugated | 87.0 | 81.2 |
| 761-35-02 | 3.3 | 0.08 | 2.22 | 0.63 | Corrugated | 96.3 | 85.8 |
| 761-35-03 | 6.7 | 0.07 | 2.19 | 0.58 | Corrugated | 91.9 | 92.5 |
| 761-35-04 | 13.3 | 0.09 | 2.01 | 0.60 | Corrugated | 90.7 | 93.4 |
| 761-35-05 | 0 | 0.18 | 1.74 | 0.74 | Corrugated | 92.6 | 88.3 |
| 761-35-06 | 3.3 | 0.10 | 1.69 | 0.53 | Corrugated | 95.6 | 94.4 |
| 761-35-07 | 6.7 | 0.11 | 1.58 | 0.52 | Corrugated | 94.9 | 94.4 |
| 761-35-08 | 13.3 | 0.11 | 1.49 | 0.49 | Corrugated | 95.1 | 93.7 |
| 761-35-09 | 6.7 | 0.11 | 1.58 | 0.52 | Corrugated | 99.4 | 91.1 |

### Example 15: Impact of Calculated Median Aerodynamic Size of Primary Particles and Particle Morphology on TLD

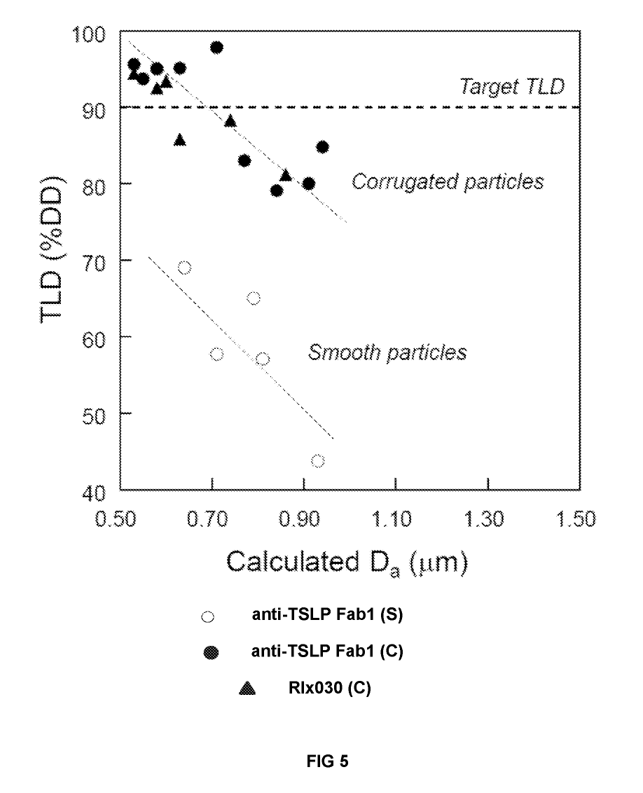

The impact of the calculated median aerodynamic diameter of primary particles, Dₐ, on the TLD is presented in FIG. 5. Particles with a smooth morphology exhibit TLD<70% of the DD that decreases rapidly with increases in Dₐ. Particles with a corrugated morphology exhibit high TLD (>80% of the DD), which increases to >90% of the DD when Dₐ is <0.7 μm.

The various features and embodiments of the present invention, referred to in individual sections above apply, as appropriate, to other sections, mutatis mutandis. Consequently features specified in one section may be combined with features specified in other sections, as appropriate.

Those skilled in the art will recognize, or be able to ascertain using no more than routine experimentation, many equivalents to the specific embodiments of the invention described herein. Such equivalents are intended to be encompassed by the following claims.

1. A carrier-free pharmaceutical powder composition comprising particles deliverable from a dry powder inhaler, comprising active agent, wherein an in vitro total lung dose is greater than 90% of a delivered dose, and wherein the particles in the delivered dose have an inertial parameter between 120 and 400 μm² L/min.

2. A carrier-free pharmaceutical composition deliverable from a dry powder inhaler, the composition comprising a plurality of particles, comprising:
a core comprising an active agent and at least one glass forming excipient, and a shell comprising hydrophobic excipient and a buffer; and
wherein the in vitro total lung dose is greater than 90% w/w of a delivered dose.

3.-4. (canceled)

5. A carrier-free pharmaceutical composition comprising a plurality of primary particles and particle agglomerates deliverable from a dry powder inhaler, the composition comprising active agent, and wherein an in vitro total lung dose is greater than 80% of a nominal dose, and wherein the primary particles are characterized by:
a corrugated morphology;
a median aerodynamic diameter between 0.3 and 1.0 μm, and wherein;
the particles and particle agglomerates delivered from a dry powder inhaler have a mass median aerodynamic diameter between 1.5 and 3.0 μm.

6. The pharmaceutical composition of any preceding claim and further including a receptacle for containing the primary particles, the receptacle suitable for containing the particles prior to their aerosolization within a dry powder inhaler, and wherein an aerosol comprising respirable agglomerates is formed upon said aerosolization.

7. A pharmaceutical powder formulation for pulmonary delivery, the powder comprising particles comprising:
1 to 100 wt % of an active agent, wherein the powder, is characterized by at least two of: a particle size distribution of at least 50% between 1 to 1.5 microns, a powder density of 0.05 to 0.3 g/cm³, an aerodynamic diameter of less than 2 microns, and a rugosity of 1.5 to 20;
and wherein the powder, when administered by inhalation, provides an in vitro total lung dose of greater than 80%.

8. (canceled)

9. The pharmaceutical powder formulation of claim 5, wherein the powder is packaged in a receptacle for use with a dry powder inhaler, and wherein when aerosolized using said dry powder inhaler, the powder is characterized by respirable agglomerates having a mass median aerodynamic diameter of less than 2 microns.

10. A pharmaceutical powder formulation for inhalation comprising particles made by a process comprising:
preparing a solution of an active agent in a water and ethanol mixture, wherein the ethanol is present between 1 and 20% and a ratio of ethanol to total solids is between 1 and 20;
spray drying the solution to obtain particulates, wherein the particulates are characterized by a particle density of 0.2 g/cm³ or lower, a geometric diameter of 1-3 microns and an aerodynamic diameter of 1 to 2 microns; and wherein
the powder, when administered by inhalation, provides an in vitro total lung dose greater than about 80%.

11.-15. (canceled)

16. A method of delivering to the lungs of a subject particles comprising a dry powder, the method comprising:
a. preparing a solution of an active agent in a mixture of water and ethanol, wherein the ethanol is present between 5 and 20%,
b. spray drying the solution to obtain a powder comprising particulates, wherein the particulates are characterized by a particle density of between about 0.05 and 0.3 g/cm³ a geometric diameter of 1-3 microns and an aerodynamic diameter of 1-2 microns;
c. packaging the spray-dried powder in a receptacle;
d. providing an inhaler having a means for extracting the powder from the receptacle, the inhaler further having a powder fluidization and aerosolization means, the inhaler operable over a patient-driven inspiratory effort of 2 to 6 kPa; the inhaler and powder together providing an inertial parameter of between 120 and 400 μm² L/min and wherein the powder, when administered by inhalation, provides at least 90% lung deposition.

17. A method of preparing a dry powder medicament formulation for pulmonary delivery, the method comprising
a. preparing a solution of an active agent in a mixture of water and ethanol, wherein the ethanol is present between 5 and 20%,
b. spray drying the solution to obtain a powder comprising particulates, wherein the particulates are characterized by a particle density of between about 0.05 and 0.3, a geometric diameter of 1-3 microns and an aerodynamic diameter of 1-2 microns; and
c. packaging the spray-dried powder in a receptacle.

18. A powder pharmaceutical composition deliverable from a dry powder inhaler, comprising particles comprising active agent, wherein an in vitro total lung dose is greater than 90% w/w of a delivered dose, and wherein the composition comprises at least one characteristic of being carrier-free, a particle density of 0.05 to 0.3 g/cm³; a particle rugosity of 3 to 20; particles made by a process comprising spray drying from an ethanol:water mixture; and particles made by a process comprising spray drying from an ethanol:water mixture having an ethanol:solids ratio of between 1 and 20.

19.-26. (canceled)
